(19) United States
(12) Patent Application Publication
(10) Pub. No.: US 2001/0049373 A1
(43) Pub. Date: Dec. 6, 2001

(54) Materials and methods for killing nematodes and nematode eggs

(76) Inventor: Richard R. Chalquest, La Jolla, CA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 09/770,121

(22) Filed: Jan. 26, 2001

Related U.S. Application Data
(60) Provisional application No. 60/179,005, filed on Jan. 28, 2000.

(51) Int. Cl. A61K 31/506; A61K 31/513; A01N 43/54; A01N 43/82

(52) U.S. Cl. 514/269; 514/252.14; 514/364

(57) ABSTRACT

Methods and compositions for the control of nematodes are disclosed. Specifically, the subject anthelmintic compounds (substituted 4-phenoxy-6-amino- pyrimidines) have been found to advantageously control animal parasitic nematodes and plant parasitic nematodes at concentrations which are non-phytotoxic. The anthelmintic compounds can be used in conjunction with other nematicidal agents such as B.t.s, avermectins, ivermectin, and milbemycin.

### CROSS-REFERENCE TO A RELATED APPLICATION

This application claims the benefit of U.S. Provisional Application No. 60/179,005, filed Jan. 28, 2000.

### BACKGROUND OF THE INVENTION

Nematodes are important plant pests which cause millions of dollars of damage each year to turf grasses, ornamental plants, and food crops. Efforts to eliminate or minimize damage caused by nematodes in agricultural settings have typically involved the use of soil fumigation with materials such as chloropicrin, methyl bromide, and dazomet, which volatilize to spread the active ingredient throughout the soil. Such fumigation materials can be highly toxic and may create an environmental hazard. Various non-fumigant chemicals have also been used, but these, too, create serious environmental problems and can be highly toxic to humans.

The accepted methodology for control of nematodes afflicting animals has centered around the use of the drug benzimidazole and its congeners. The use of these drugs on a wide scale has led to many instances of resistance among nematode populations (Prichard, R. K. et al. [1980] “The problem of anthelmintic resistance in nematodes,” Austr. Vet. J. 56:239-251; Coles, G. C. [1986] “Anthelmintic resistance in sheep,” In Veterinary Clinics of North America: Food Animal Practice, Vol 2:423-432 [Herd, R. P., Eds.] W. B. Saunders, New York).

The pesticidal activity of avermectins is well known. The avermectins are disaccharide derivatives of pentacyclic, 16-membered lactones. They can be divided into four major compounds: A₁ₐ, A₂ₐ, B₁ₐ, and B₂ₐ; and four minor compounds: A_{1b}, A_{2b}, B_{1b}, and B_{2b}.

The organism which produces avermectins was isolated and identified as Streptomyces avermitilis MA-4680 (NRRL-8165). Characteristics of the avermectin producing culture and the fermentation process are well documented and known to those skilled in the art (Burg, R. W. et al. [1979] “Avermectins, New Family of Potent Anthelmintic Agents: Producing Organism and Fermentation,” Antimicrob. Agents Chemother. 15(3):361-367). The isolation and purification of these compounds is also described in U.S. Pat. No. 4,310,519, issued Jan. 12, 1982.

Another family of pesticides produced by fermentation are the milbemycins, which are closely related to the avermectins. The milbemycins can be produced by a variety of Streptomyces and originally differed from the avermectins only in the C-13 position. The milbemycins and their many derivatives are also well known to those skilled in the art and are the subject of U.S. patents. See, for example, U.S. Pat. No. 4,547,520.

While the avermectins were initially investigated for their anthelmintic activities, they were later found to have other insecticidal properties, although the degree varies. The activity of avermectins must generally be determined empirically.

22,23-dihydroavermectin B₁ is a synthetic derivative of the avermectins and has been assigned the nonproprietary name of ivermectin. It is a mixture of 80% 22,23-dihydroavermectin B₁ₐ and 20% 22,23-dihydroavermectin B_{1b}. Ivermectin has been tested on a variety of laboratory and domestic animals for control of nematodes, ticks, and heartworms.

Avermectin B₂ₐ is active against the root-knot nematode, Meloidogyne incognita. It is reported to be 10-30 times as potent as commercial contact nematicides when incorporated into soil at 0.16-0.25 kg/ha (Boyce Thompson Institute for Plant Research 58th Annual Report [1981]; Putter, I. et al. [1981] “Avermectins: Novel Insecticides, Acaracides, and Nematicides from a Soil Microorganism,” Experientia 37:963-964). Avermectin B₂ₐ is not toxic to tomatoes or cucumbers at rates of up to 10 kg/ha. Avermectin B₁ is a combination of avermectin B₁ₐ (major component) and avermectin B_{1b}. It has demonstrated a broad spectrum of insecticidal activities. The data indicate that avermectin B₁ is primarily a miticide, although it is also effective on the Colorado potato beetle, potato tuberworm, beet armyworm, diamondback moth, gypsy moth, and the European corn borer.

The use of avermectins in various agricultural applications has been described in publications and patents. The use of avermectin with spray oils (lightweight oil compositions) has been described. See, for example, U.S. Pat. No. 4,560,677 issued Dec. 24, 1985; EPO applications 0 094 779 and 0 125 155; and Anderson, T. E., J. R. Babu, R. A. Dybas, H. Mehta (1986) J. Econ. Entomol. 79:197-201.

There is a continuing need for new, alternative materials and methods useful for killing nematodes.

### BRIEF SUMMARY OF THE INVENTION

The subject invention concerns compositions and processes for controlling nematodes. In one embodiment, the subject invention comprises the use of substituted 4-phenoxy-6-amino-pyrimidine compounds to control nematodes which infest and afflict animals. Nematodes which infest plants or the situs of plants can also be controlled using the methods and compositions of the subject invention, as can other acarid and arthropod pests.

Preferred compounds useful according to the subject invention can be represented by the Formulae I, II, III, IV, and V as further described herein.

1. A urea derivative of the following Formula I:

Ar—(Alk)₀₋₁—NH—CO—NR¹—Alk—R² (Formula I)

wherein Ar is aryl or heteroaryl optionally substituted by one or more R³ groups;

each Alk is a linear or cyclic alkylene radical of up to 8 C atoms;

R¹ is H or C,₆ alkyl;

R² is heteroaryl or heterocycloalkyl optionally substituted by Ar, or forms such a group by cyclisation with R¹; and

R³ is OH, halogen, CF₃, OCF, or a group selected from NH₂, SO₂—C₁₋₆ alkyl, C₆₋₁₀ aryl, C₆₋₁₀ aryloaxy, C₅₋₆ cycloalkyl, C₁₋₅ alkoxy, and C₁₋₆ alkyl, said group being optionally substituted by OH, C₁₋₆ alkoxy, C₁₋₆ alkyl, phenyl, halogen, or CF₃.

Particularly preferred anthelmintic compounds according to Formula I are exemplified herein by compounds represented by structures 1-10 (depicted in FIGS. 1-10, respectively), which have been assigned the respective reference numbers:

| | |
|---|---|
| AKC 111 | (STRUCTURE 1), |
| AKC 112 | (STRUCTURE 2), |
| AKC 113 | (STRUCTURE 3), |
| AKC 107 | (STRUCTURE 4), |
| AKC 114 | (STRUCTURE 5), |
| AKC 108 | (STRUCTURE 6), |
| AKC 115 | (STRUCTURE 7), |
| AKC 116 | (STRUCTURE 8), |
| AKC 117 | (STRUCTURE 9), and |
| AKC 118 | (STRUCTURE 10). |

2. A heterocycle-substituted amide of the following Formula II:

Ar—(Alk)₀₋₁—NH—CO—Het (Formula II)

wherein Ar is aryl or heteroaryl optionally substituted by one or more R³ groups;

each Alk is an optionally cyclic alkylene radical of up to 8 C atoms;

Het is heteroaryl or heterocycloalkyl optionally substituted by Ar and/or R³; and

R³ is OH, halogen, CF₃, OCF₃, or a group selected from NH₂, SO₂ alkyl, C₆₋₁₀ aryl, C₁₋₆ alkoxy, and C₁₋₆ alkyl, said group being optionally substituted by OH, C₁₋₆ alkoxy, C₁₋₆ alkyl, phenyl, halogen, or CF₃.

Particularly preferred anthelmintic compounds according to Formula II are exemplified herein by compounds represented by structures 11-25 (depicted in FIGS. 11-25 respectively), which have been assigned the respective reference numbers:

| | |
|---|---|
| AKC 119 | (STRUCTURE 11), |
| AKC 110 | (STRUCTURE 12), |
| AKC 120 | (STRUCTURE 13), |
| AKC 121 | (STRUCTURE 14), |
| AKC 2153 | (STRUCTURE 15), |
| AKC 122 | (STRUCTURE 16), |
| AKC 104 | (STRUCTURE 17), |
| AKC 123 | (STRUCTURE 18), |
| AKC 124 | (STRUCTURE 19), |
| AKC 125 | (STRUCTURE 20), |
| AKC 105 | (STRUCTURE 21), |
| AKC 126 | (STRUCTURE 22), |
| AKC 102 | (STRUCTURE 23), |
| AKC 103 | (STRUCTURE 24), and |
| AKC 171 | (STRUCTURE 25). |

3. A secondary arylamine of the following Formula III:

Ar—NH—CHR—CH₂—CO—Y (Formula III)

wherein Ar is aryl or heteroaryl optionally substituted by one or more R³ groups;

R is aryl, heteroaryl, or heterocycloalkyl optionally substituted by R³;

Y is C₁₋₆ alkyl, aryl, or heteroaryl optionally substituted by R³;

or R and Y together form a cycloalkyl or heterocycloalkyl ring; and

R³ is OH, halogen, CF₃, OCF₃, or a group selected from NH₂, SO₂ alkyl C₆₋₁₀ aryl, C₁₋₆ alkoxy, and C₁₋₆ alkyl, said group being optionally substituted by OH, C₁₋₆ alkoxy, C₁₋₆ alkyl, phenyl, halogen, or CF₃.

Particularly preferred anthelmintic compounds according to Formula III are exemplified herein by compounds represented by structures 26-31 (depicted in FIGS. 26-31, respectively), which have been assigned the respective reference numbers:

| | |
|---|---|
| AKC 128 | (STRUCTURE 26), |
| AKC 129 | (STRUCTURE 27), |
| AKC 130 | (STRUCTURE 28), |
| AKC 131 | (STRUCTURE 29), |
| AKC 132 | (STRUCTURE 30), and |
| AKC 133 | (STRUCTURE 31). |

4. A diaryl amine of the following Formula IV:

Ar—(Z)₀₋₁—Ar—(CH₂)₀₋₁—NHR (Formula IV)

wherein Ar is aryl or heteroaryl optionally substituted by one or more R³ groups;

Z is NH, O, S, or Alk; and Alk is a linear or cyclic alkylene radical of up to 8 C atoms

wherein said radical optionally includes one or more heteroatoms;

R is H or R³,

R³ is OH, halogen, CF₃, OCF₃, or a group selected from NH₂, SO₂ alkyl, C₆₋₁₀ aryl, C₁₋₆ alkoxy, and C₁₋₆ alkyl, said group being optionally substituted by OH, C₁₋₆ alkoxy, C₁₋₆ alkyl, phenyl, halogen, or CF₃.

Particularly preferred anthelmintic compounds according to Formula IV are exemplified by compounds represented by structures 32-37 (depicted in FIGS. 32-37, respectively), which have been assigned the respective reference numbers:

| | |
|---|---|
| AKC 109 | (STRUCTURE 32), |
| AKC 134 | (STRUCTURE 33), |
| AKC 135 | (STRUCTURE 34), |
| AKC 136 | (STRUCTURE 35), |
| AKC 137 | (STRUCTURE 36), and |
| AKC 138 | (STRUCTURE 37). |

5. A substituted heteropolycyclic compound of the following Formula V:

Het₂—Q (Formula V)

wherein Het, is two or three fused aromatic rings including one or more heteroatoms selected from N, O and S, and Q includes at least one substituent selected from OH, COOR³ and CONHR³, and optionally also another substituent selected from alkyl and alkenyl of up to 10 C atoms;

wherein R³is OH, halogen, CF₃, OCF₃, or a group selected from NH₂, SO₂ alkyl, C₆₋₁₀ aryl, C₁₋₆ alkoxy, and C₁₋₆ alkyl, said group being optionally substituted by OH, C₁₋₆ alkoxy, C₁₋₆ alkyl, phenyl, halogen, or CF₃.

Particularly preferred anthelmintic compounds according to Formula V are exemplified by compounds represented by structures 38-43 (depicted in FIGS. 38-43, respectively), which have been assigned the respective reference numbers:

| | |
|---|---|
| AKC 139 | (STRUCTURE 38), |
| AKC 140 | (STRUCTURE 39), |
| AKC 141 | (STRUCTURE 40), |
| AKC 142 | (STRUCTURE 41), |
| AKC 143 | (STRUCTURE 42), and |
| AKC 144 | (STRUCTURE 43). |

For the foregoing Formulae I, II, III, IV, and V, as well as throughout this disclosure, the following definitions apply.

“Aryl” refers to an aromatic group, typically of 6-10 C atoms, such as phenyl or naphthyl.

“Alk” includes, for example, (CH₂), wherein n is an integer of up to 6, e.g. 1, 2, 3, or 4, or cyclohexylene.

“Heteroaryl” means an aromatic group including one or more heteroatoms selected from O, S and N. It will typically have 5 or 6 ring atoms. It may also be fused to one or more aryl groups. Examples are in the illustrated compounds.

“Heterocycloalkyl” means a cycloalkyl group in which one or more C atoms are replaced by one or more heteroatoms selected from O, S and N. It will typically have 5 or 6 ring atoms. Examples are in the illustrated compounds of structures 1-43.

Other preferred anthelmintic compounds useful according to the subject invention are represented by structures 44, 45, and 46 (depicted in FIGS. 44-46, respectively),and have been assigned the respective reference numbers:

| | |
|---|---|
| AKC 145 | (STRUCTURE 44), |
| AKC 146 | (STRUCTURE 45), and |
| AKC 147 | (STRUCTURE 46). |

The invention process is particularly valuable to control nematodes which are pests to animals, as well as nematodes attacking the roots of desired crop plants, ornamental plants, and turf grasses. The desired crop plants can be, for example, cotton, soybean, tomatoes, potatoes, grapes, strawberries, bananas or vegetables.

In one embodiment of the subject invention, the subject anthelmintic compounds are used in conjunction with one or more other nematicidal agents. The other nematicidal agents may be, for example, a biological agent, an avermectin, a milbemycin, or a fatty acid.

In another embodiment, the subject invention further provides methods for killing the eggs of nematodes. Thus, the subject invention further relates to the surprising discovery that certain compounds have ovicidal activity against nematode eggs. Compositions comprising the anthelmintic compounds of the subject invention are particularly useful for preplant applications in nematode-control schemes.

### DESCRIPTION OF THE DRAWINGS

FIG. 1 depicts Structure 1 which represents anthelmintic compound AKC 111.

FIG. 2 depicts Structure 2 which represents anthelmintic compound AKC 112.

FIG. 3 depicts Structure 3 which represents anthelmintic compound AKC 113.

FIG. 4 depicts Structure 4 which represents anthelmintic compound AKC 107.

FIG. 5 depicts Structure 5 which represents anthelmintic compound AKC 114.

FIG. 6 depicts Structure 6 which represents anthelmintic compound AKC 108.

FIG. 7 depicts Structure 7 which represents anthelmintic compound AKC 115.

FIG. 8 depicts Structure 8 which represents anthelmintic compound AKC 116.

FIG. 9 depicts Structure 9 which represents anthelmintic compound AKC 117.

FIG. 10 depicts Structure 10 which represents anthelmintic compound AKC 118.

FIG. 11 depicts Structure 11 which represents anthelmintic compound AKC 119.

FIG. 12 depicts Structure 12 which represents anthelmintic compound AKC 110.

FIG. 13 depicts Structure 13 which represents anthelmintic compound AKC 120.

FIG. 14 depicts Structure 14 which represents anthelmintic compound AKC 121.

FIG. 15 depicts Structure 15 which represents anthelmintic compound AKC 2153.

FIG. 16 depicts Structure 16 which represents anthelmintic compound AKC 122.

FIG. 17 depicts Structure 17 which represents anthelmintic compound AKC 104.

FIG. 18 depicts Structure 18 which represents anthelmintic compound AKC 123.

FIG. 19 depicts Structure 19 which represents anthelmintic compound AKC 124.

FIG. 20 depicts Structure 20 which represents anthelmintic compound AKC 125.

FIG. 21 depicts Structure 21 which represents anthelmintic compound AKC 105.

FIG. 22 depicts Structure 22 which represents anthelmintic compound AKC 126.

FIG. 23 depicts Structure 23 which represents anthelmintic compound AKC 102.

FIG. 24 depicts Structure 24 which represents anthelmintic compound AKC 103.

FIG. 25 depicts Structure 25 which represents anthelmintic compound AKC 171.

FIG. 26 depicts Structure 26 which represents anthelmintic compound AKC 128.

FIG. 27 depicts Structure 27 which represents anthelmintic compound AKC 129.

FIG. 28 depicts Structure 28 which represents anthelmintic compound AKC 130.

FIG. 29 depicts Structure 29 which represents anthelmintic compound AKC 121.

FIG. 30 depicts Structure 30 which represents anthelmintic compound AKC 132.

FIG. 31 depicts Structure 31 which represents anthelmintic compound AKC 133.

FIG. 32 depicts Structure 32 which represents anthelmintic compound AKC 109.

FIG. 33 depicts Structure 33 which represents anthelmintic compound AKC 134.

FIG. 34 depicts Structure 34 which represents anthelmintic compound AKC 135.

FIG. 35 depicts Structure 35 which represents anthelmintic compound AKC 136.

FIG. 36 depicts Structure 36 which represents anthelmintic compound AKC 137.

FIG. 37 depicts Structure 37 which represents anthelmintic compound AKC 138.

FIG. 38 depicts Structure 38 which represents anthelmintic compound AKC 139.

FIG. 39 depicts Structure 39 which represents anthelmintic compound AKC 140.

FIG. 40 depicts Structure 40 which represents anthelmintic compound AKC 141.

FIG. 41 depicts Structure 41 which represents anthelmintic compound AKC 142.

FIG. 42 depicts Structure 42 which represents anthelmintic compound AKC 143.

FIG. 43 depicts Structure 43 which represents anthelmintic compound AKC 144.

FIG. 44 depicts Structure 44 which represents anthelmintic compound AKC 145.

FIG. 45 depicts Structure 45 which represents anthelmintic compound AKC 146.

FIG. 46 depicts Structure 46 which represents anthelmintic compound AKC 147.

FIG. 47 depicts a basic structure, Structure 47, of a preferred class of anthelmintic compound.

FIG. 48 depicts anthelmintic compound AKC 340 of the class represented in FIG. 47.

FIG. 49 depicts anthelmintic compound AKC 339 of the class represented in FIG. 47.

FIG. 50 depicts anthelmintic compound AKC 148 of the class represented in FIG. 47.

FIG. 51 depicts anthelmintic compound AKC 347 of the class represented in FIG. 47.

FIG. 52 depicts anthelmintic compound AKC 342 of the class represented in FIG. 47.

FIG. 53 depicts anthelmintic compound AKC 348 of the class represented in FIG. 47.

FIG. 54 depicts anthelmintic compound AKC 343 of the class represented in FIG. 47.

FIG. 55 depicts anthelmintic compound AKC 341 of the class represented in FIG. 47.

FIG. 56 depicts anthelmintic compound AKC 344 of the class represented in FIG. 47.

FIG. 57 depicts anthelmintic compound AKC 345 of the class represented in FIG. 47.

FIG. 58 depicts anthelmintic compound AKC 346 of the class represented in FIG. 47.

FIG. 59 depicts anthelmintic compound AKC 350 of the class represented in FIG. 47.

FIG. 60 depicts anthelmintic compound AKC 349 of the class represented in FIG. 47.

FIG. 61 depicts anthelmintic compound AKC 351 of the class represented in FIG. 47.

FIG. 62 depicts anthelmintic compound AKC 353 of the class represented in FIG. 47.

FIG. 63 depicts anthelmintic compound AKC 354 of the class represented in FIG. 47.

FIG. 64 depicts anthelmintic compound AKC 352 of the class represented in FIG. 47.

FIG. 65 depicts anthelmintic compound AKC 357 of the class represented in FIG. 47.

FIG. 66 depicts anthelmintic compound AKC 355 of the class represented in FIG. 47.

FIG. 67 depicts anthelmintic compound AKC 358 of the class represented in FIG. 47.

FIG. 68 depicts anthelmintic compound AKC 359 of the class represented in FIG. 47.

FIG. 69 depicts anthelmintic compound AKC 149 of the class represented in FIG. 47.

FIG. 70 depicts anthelmintic compound AKC 356 of the class represented in FIG. 47.

FIG. 71 depicts anthelmintic compound AKC 360 of the class represented in FIG. 47.

FIG. 72 depicts anthelmintic compound AKC 362 of the class represented in FIG. 47.

FIG. 73 depicts anthelmintic compound AKC 361 of the class represented in FIG. 47.

FIG. 74 depicts anthelmintic compound AKC 367 of the class represented in FIG. 47.

FIG. 75 depicts anthelmintic compound AKC 365 of the class represented in FIG. 47.

FIG. 76 depicts anthelmintic compound AKC 363 of the class represented in FIG. 47.

FIG. 77 depicts anthelmintic compound AKC 366 of the class represented in FIG. 47.

FIG. 78 depicts anthelmintic compound AKC 364 of the class represented in FIG. 47.

FIG. 79 depicts anthelmintic compound AKC 371 of the class represented in FIG. 47.

FIG. 80 depicts anthelmintic compound AKC 369 of the class represented in FIG. 47.

FIG. 81 depicts anthelmintic compound AKC 373 of the class represented in FIG. 47.

FIG. 82 depicts anthelmintic compound AKC 375 of the class represented in FIG. 47.

FIG. 83 depicts anthelmintic compound AKC 370 of the class represented in FIG. 47.

FIG. 84 depicts anthelmintic compound AKC 368 of the class represented in FIG. 47.

FIG. 85 depicts anthelmintic compound AKC 372 of the class represented in FIG. 47.

FIG. 86 depicts anthelmintic compound AKC 376 of the class represented in FIG. 47.

FIG. 87 depicts anthelmintic compound AKC 374 of the class represented in FIG. 47.

FIG. 88 depicts anthelmintic compound AKC 377 of the class represented in FIG. 47.

FIG. 89 depicts anthelmintic compound AKC 378 of the class represented in FIG. 47.

FIG. 90 depicts anthelmintic compound AKC 380 of the class represented in FIG. 47.

FIG. 91 depicts anthelmintic compound AKC 384 of the class represented in FIG. 47.

FIG. 92 depicts anthelmintic compound AKC 387 of the class represented in FIG. 47.

FIG. 93 depicts anthelmintic compound AKC 381 of the class represented in FIG. 47.

FIG. 94 depicts anthelmintic compound AKC 388 of the class represented in FIG. 47.

FIG. 95 depicts anthelmintic compound AKC 382 of the class represented in FIG. 47.

FIG. 96 depicts anthelmintic compound AKC 385 of the class represented in FIG. 47.

FIG. 97 depicts anthelmintic compound AKC 389 of the class represented in FIG. 47.

FIG. 98 depicts anthelmintic compound AKC 379 of the class represented in FIG. 47.

FIG. 99 depicts anthelmintic compound AKC 386 of the class represented in FIG. 47.

FIG. 100 depicts anthelmintic compound AKC 383 of the class represented in FIG. 47.

FIG. 101 depicts anthelmintic compound AKC 390 of the class represented in FIG. 47.

FIG. 102 depicts anthelmintic compound AKC 391 of the class represented in FIG. 47.

FIG. 103 depicts anthelmintic compound AKC 398 of the class represented in FIG. 47.

FIG. 104 depicts anthelmintic compound AKC 392 of the class represented in FIG. 47.

FIG. 105 depicts anthelmintic compound AKC 399 of the class represented in FIG. 47.

FIG. 106 depicts anthelmintic compound AKC 393 of the class represented in FIG. 47.

FIG. 107 depicts anthelmintic compound AKC 400 of the class represented in FIG. 47.

FIG. 108 depicts anthelmintic compound AKC 394 of the class represented in FIG. 47.

FIG. 109 depicts anthelmintic compound AKC 401 of the class represented in FIG. 47.

FIG. 110 depicts anthelmintic compound AKC 395 of the class represented in FIG. 47.

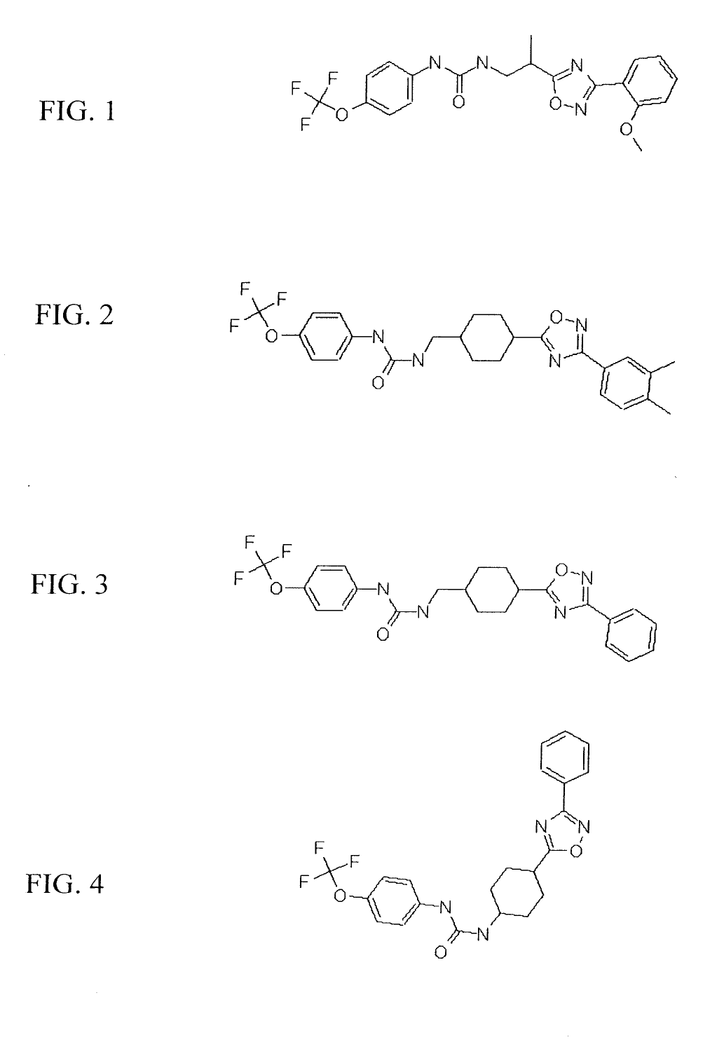

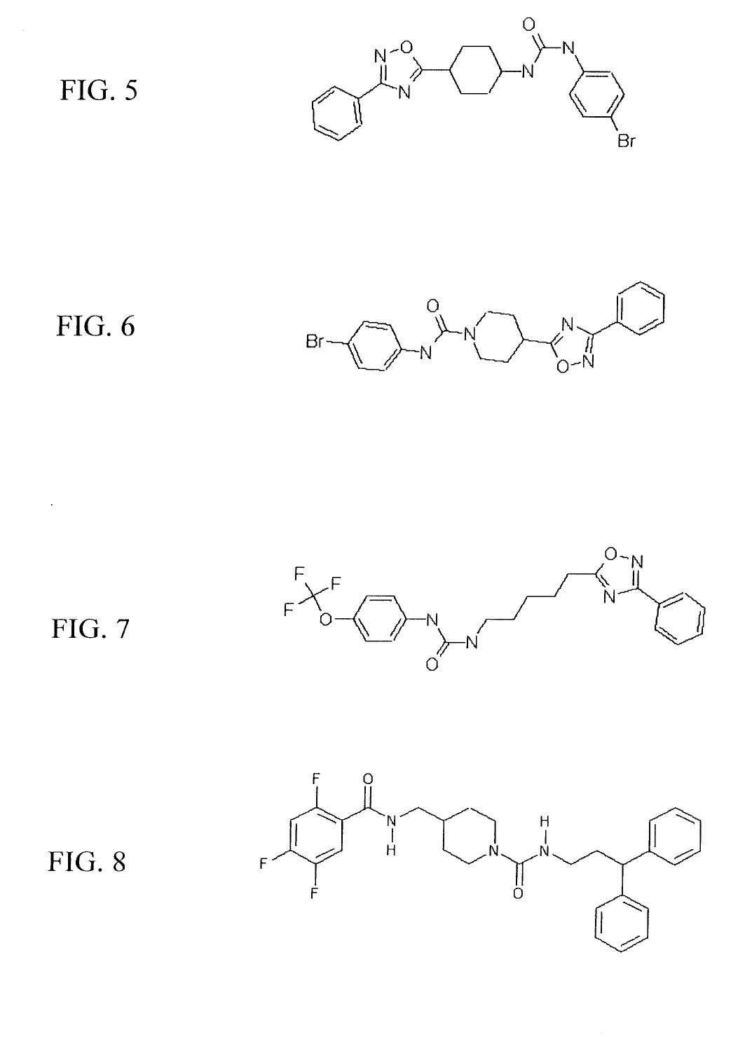

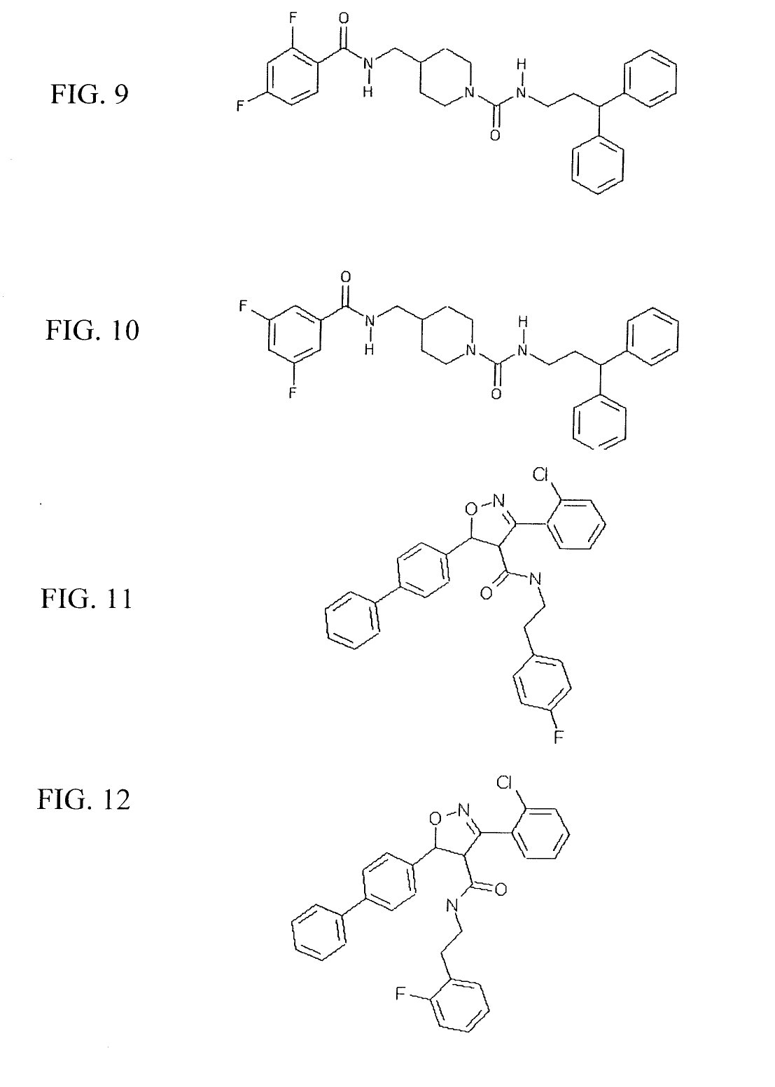

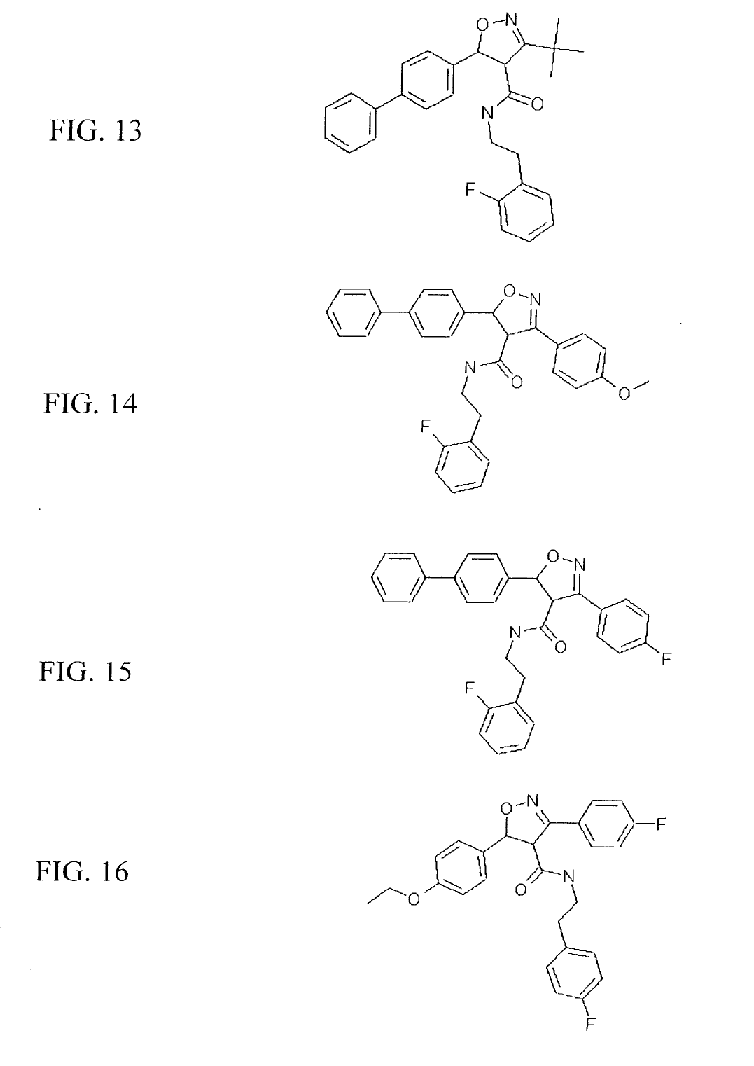

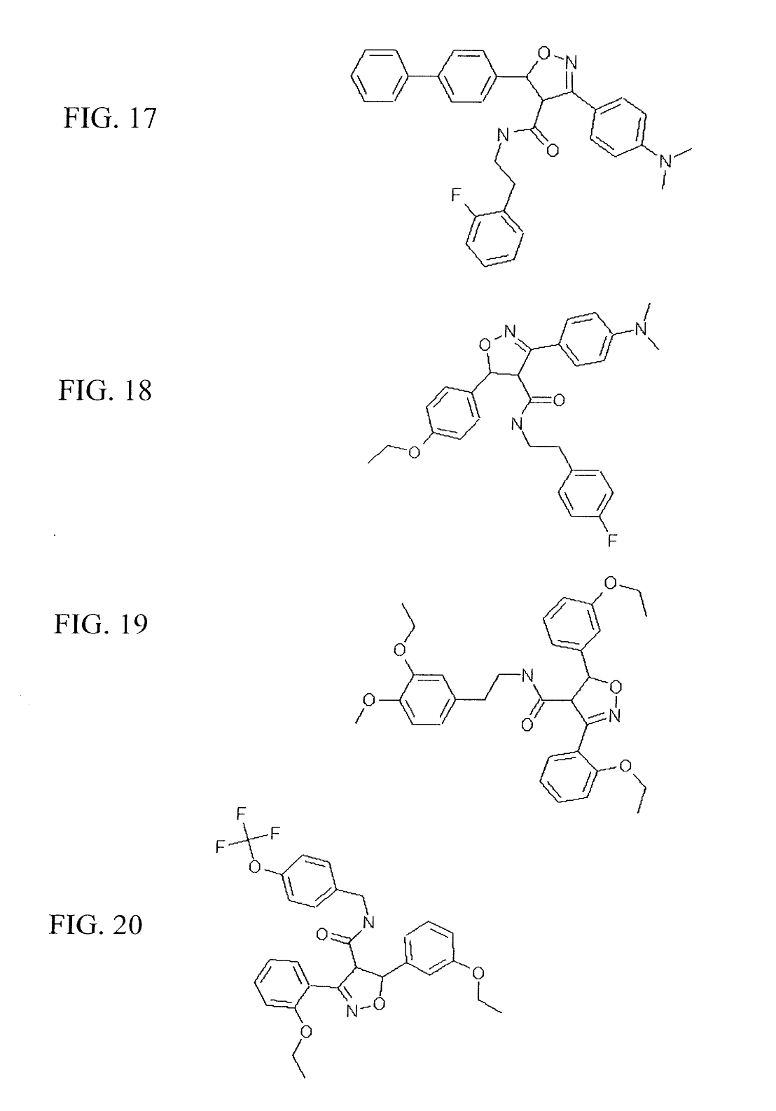

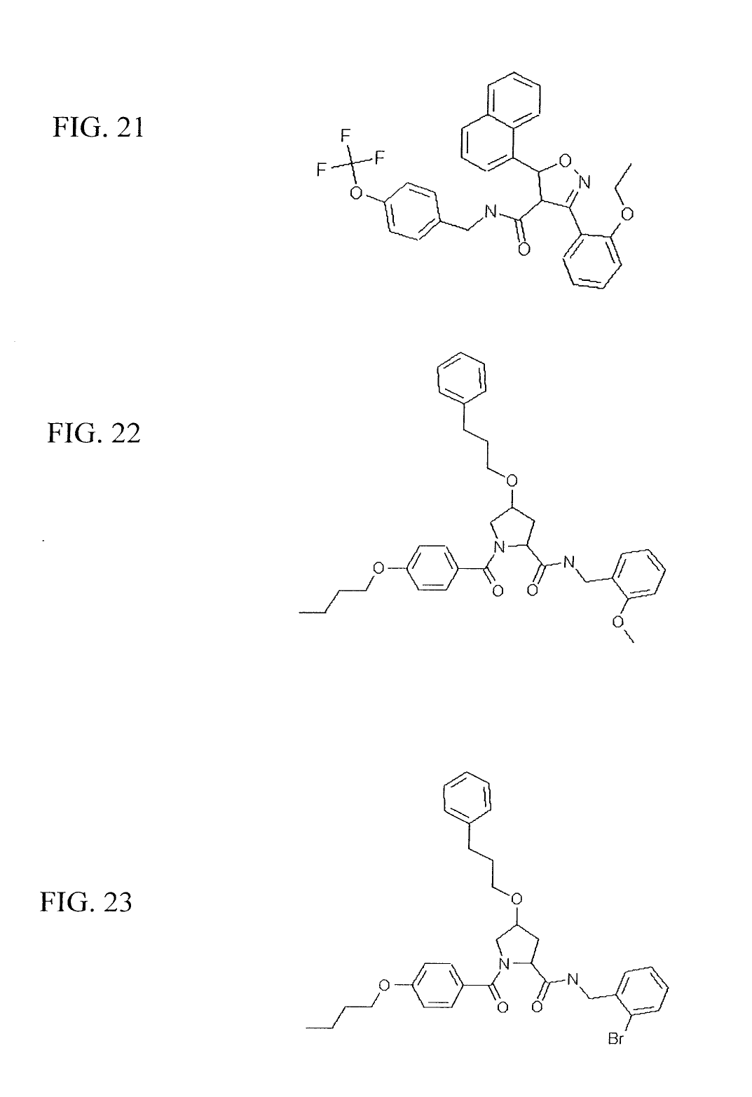

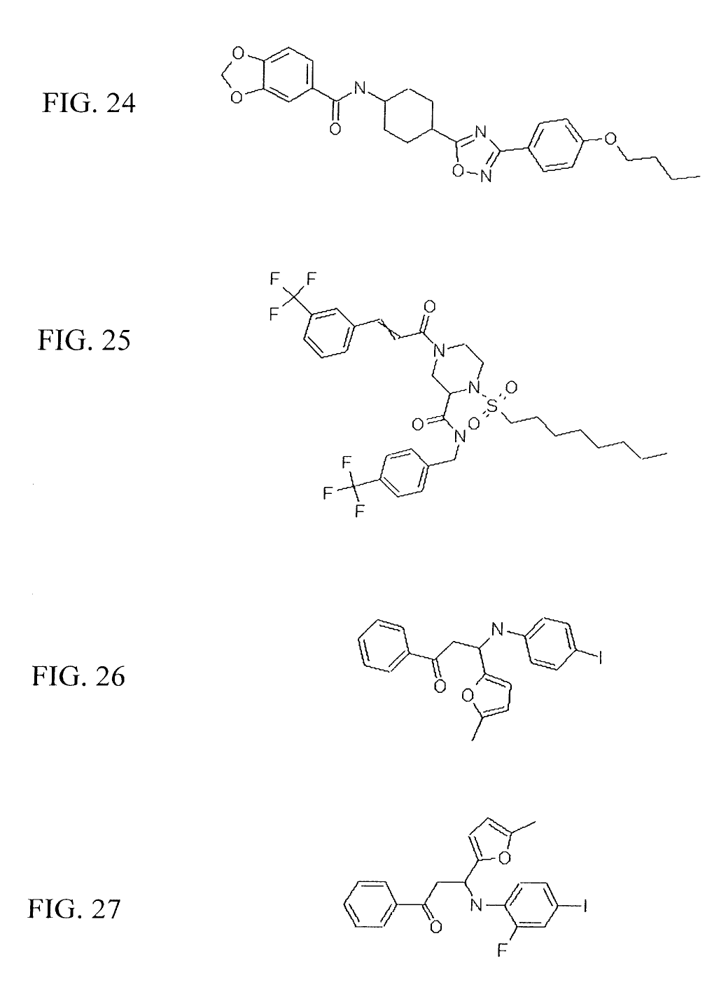

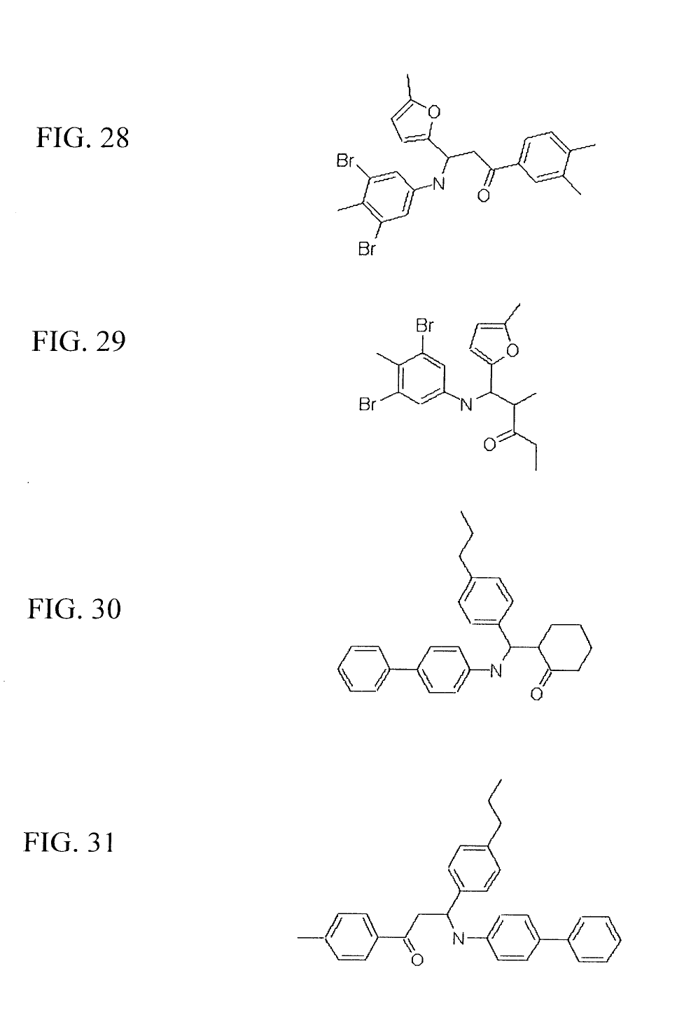

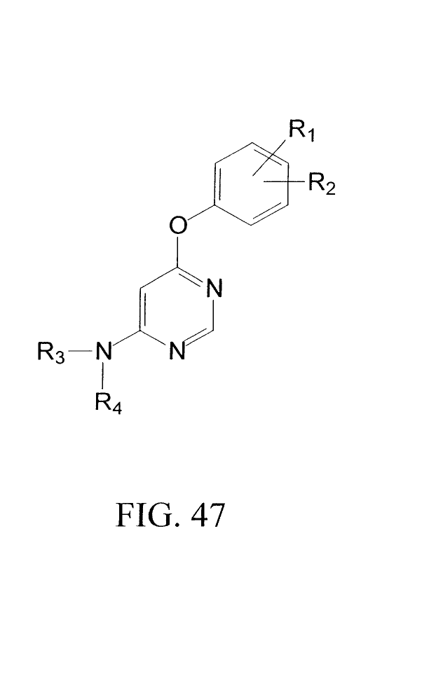

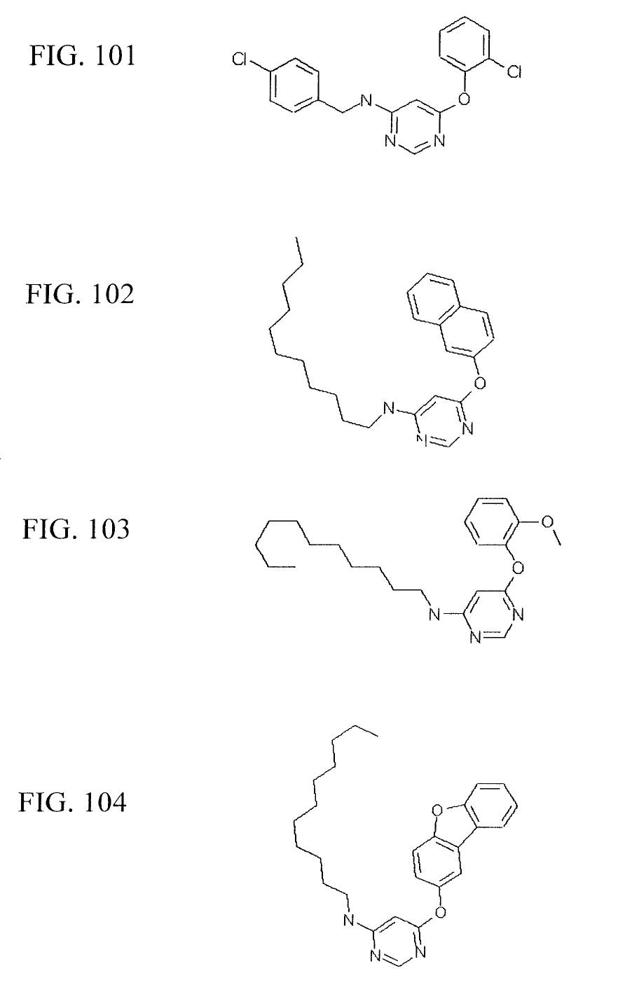

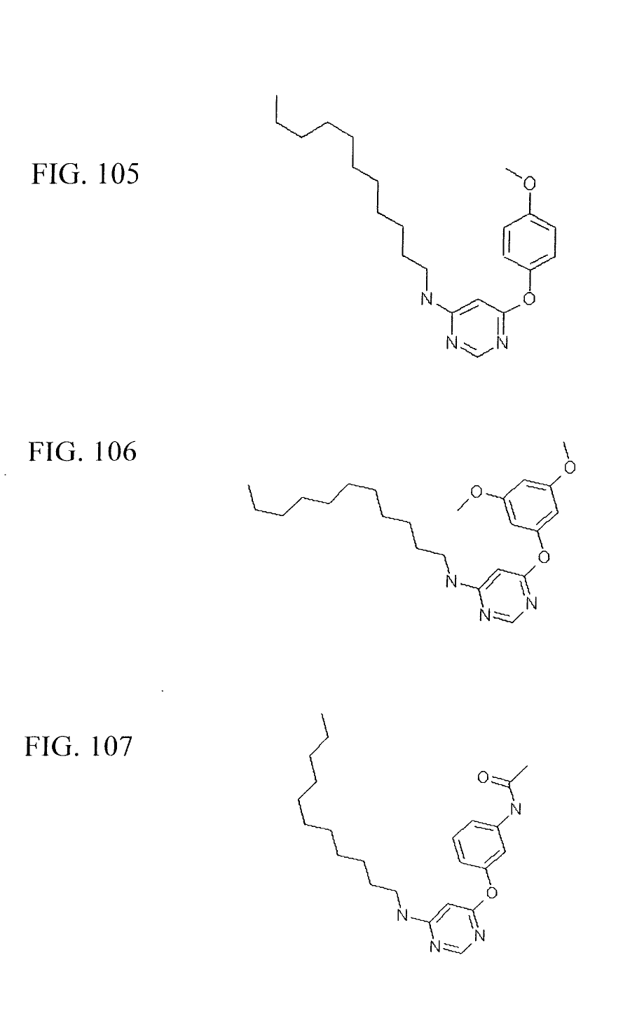

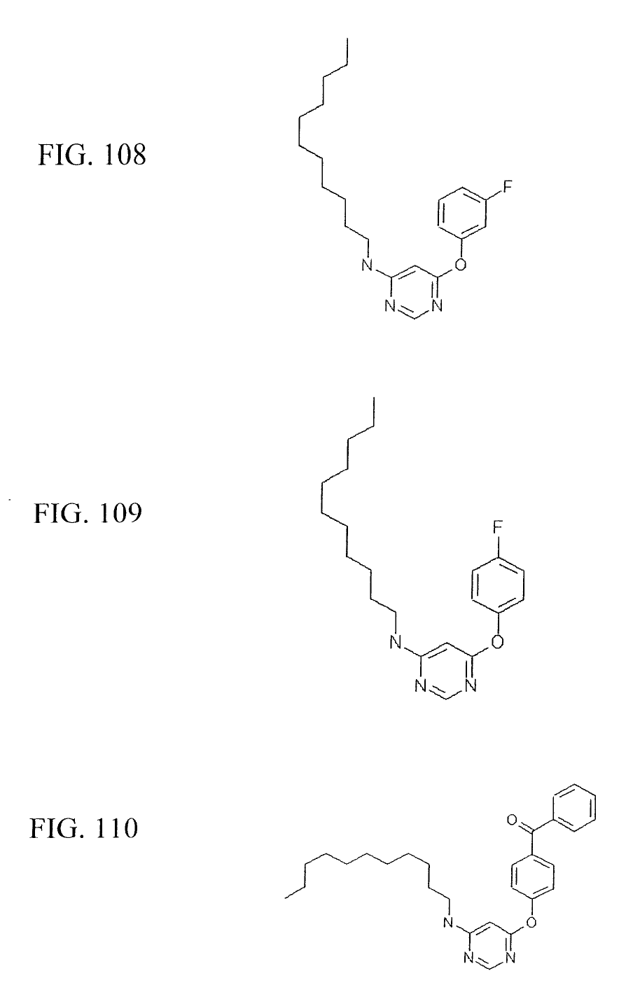

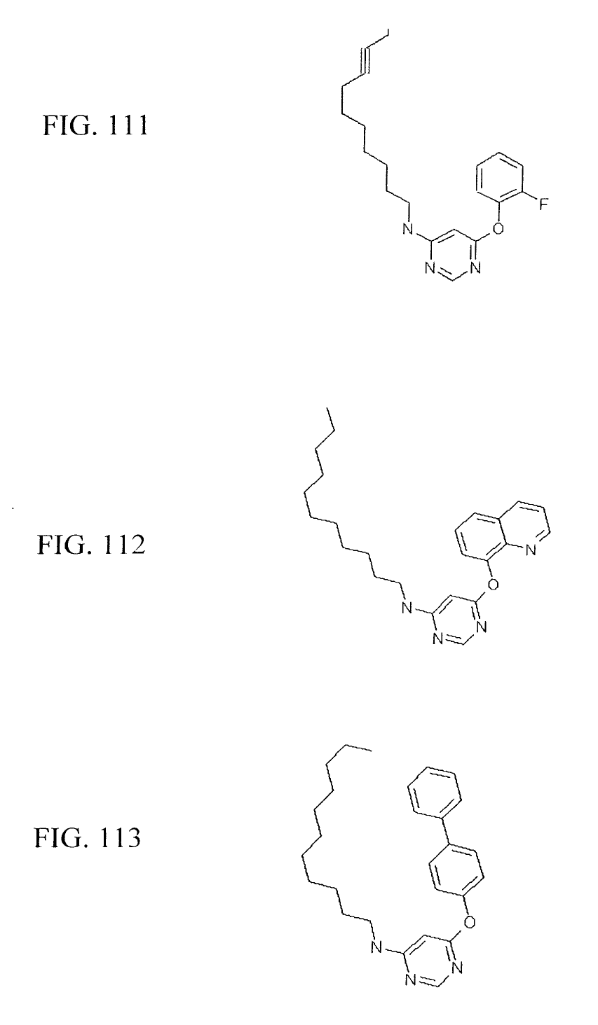

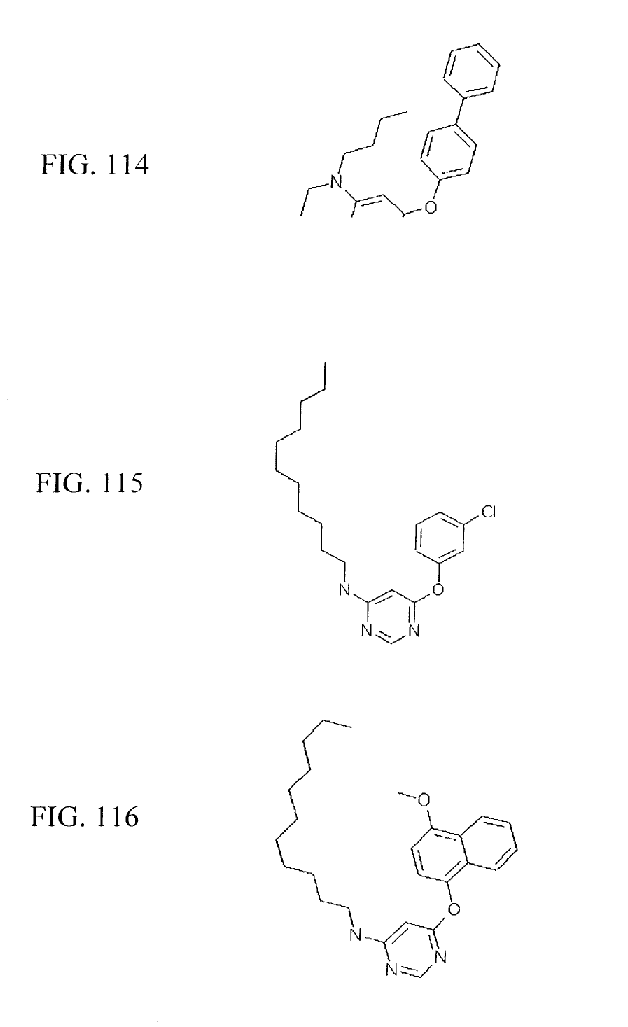

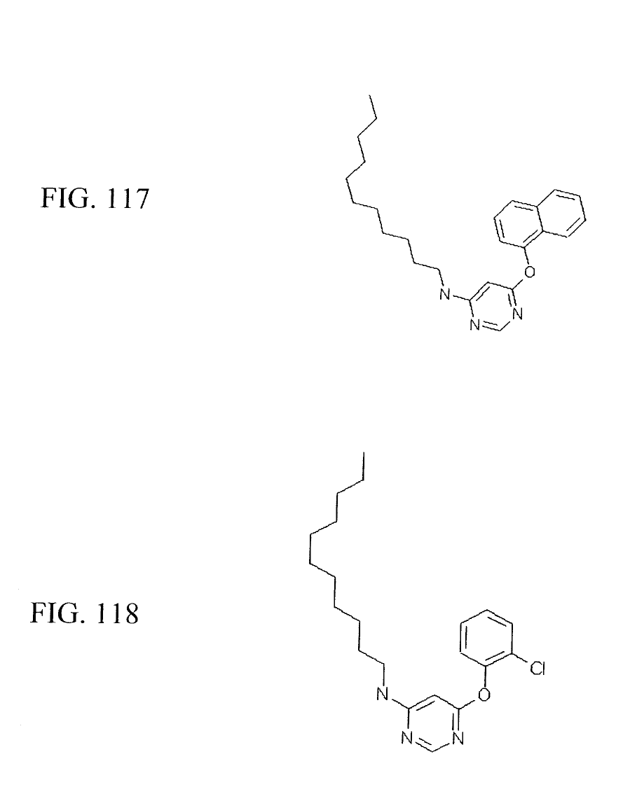

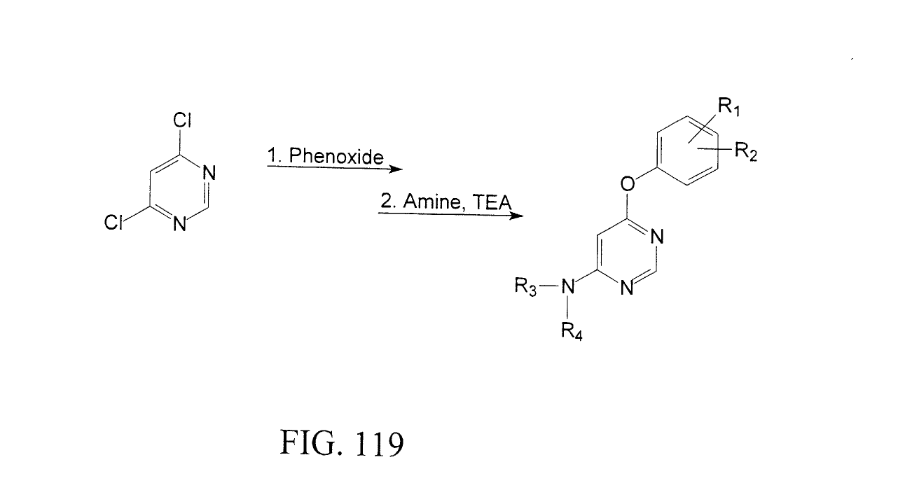

FIG. 101 depicts anthelmintic compound AKC 151 of the class represented in FIG. 47.

FIG. 112 depicts anthelmintic compound AKC 150 of the class represented in FIG. 47.

FIG. 113 depicts anthelmintic compound AKC 402 of the class represented in FIG. 47.

FIG. 114 depicts anthelmintic compound AKC 405 of the class represented in FIG. 47.

FIG. 115 depicts anthelmintic compound AKC 396 of the class represented in FIG. 47.

FIG. 116 depicts anthelmintic compound AKC 403 of the class represented in FIG. 47.

FIG. 117 depicts anthelmintic compound AKC 397 of the class represented in FIG. 47.

FIG. 118 depicts anthelmintic compound AKC 404 of the class represented in FIG. 47.

FIG. 119 depicts one library scheme by which the skilled artisan can create the compounds represented by the structure depicted in FIG. 47.

### DETAILED DISCLOSURE OF THE INVENTION

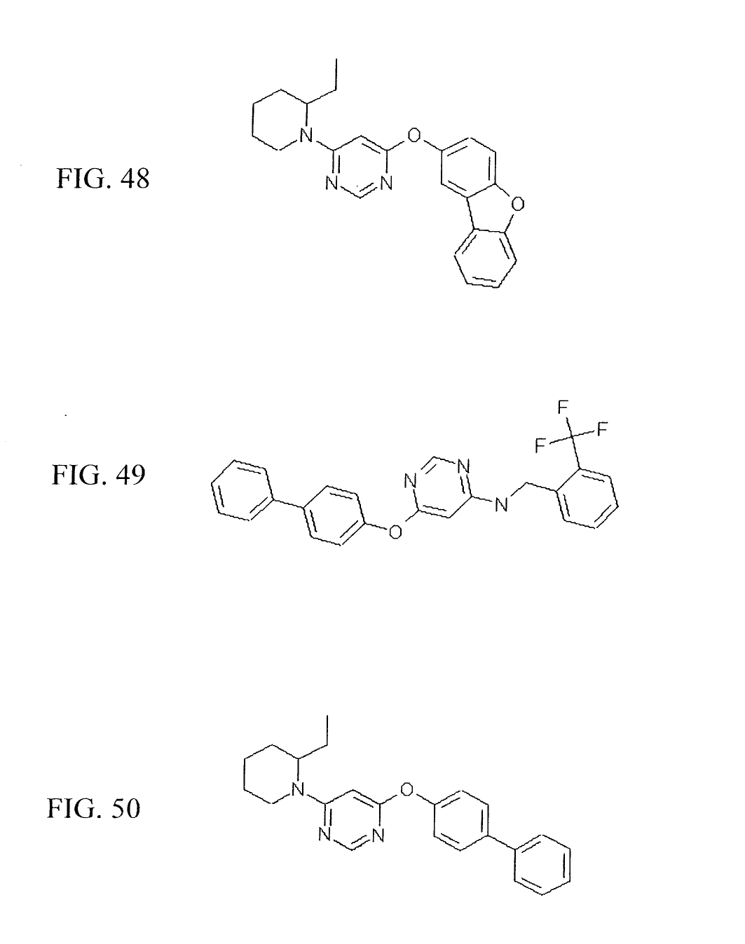

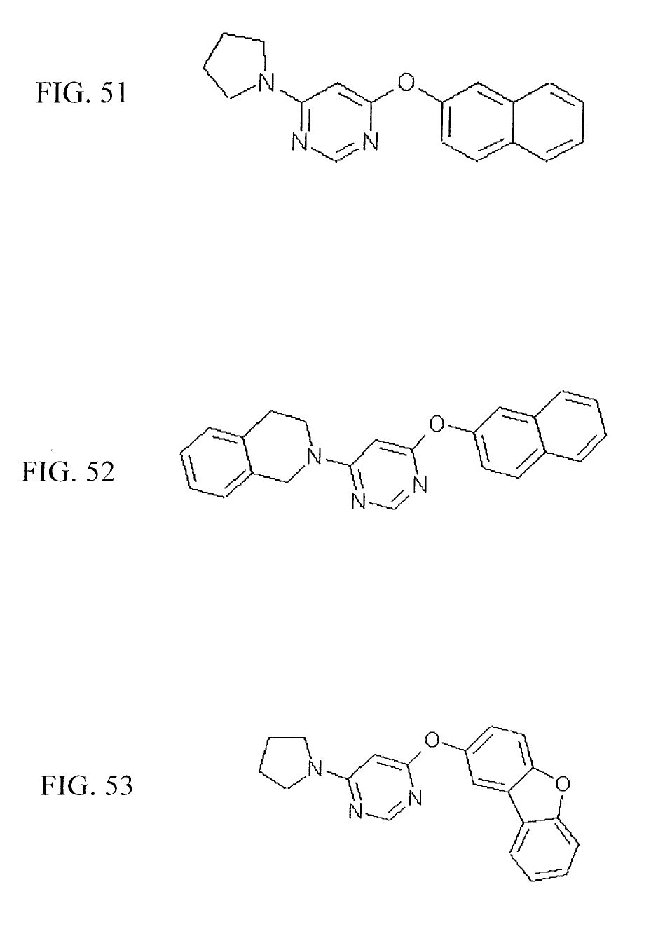

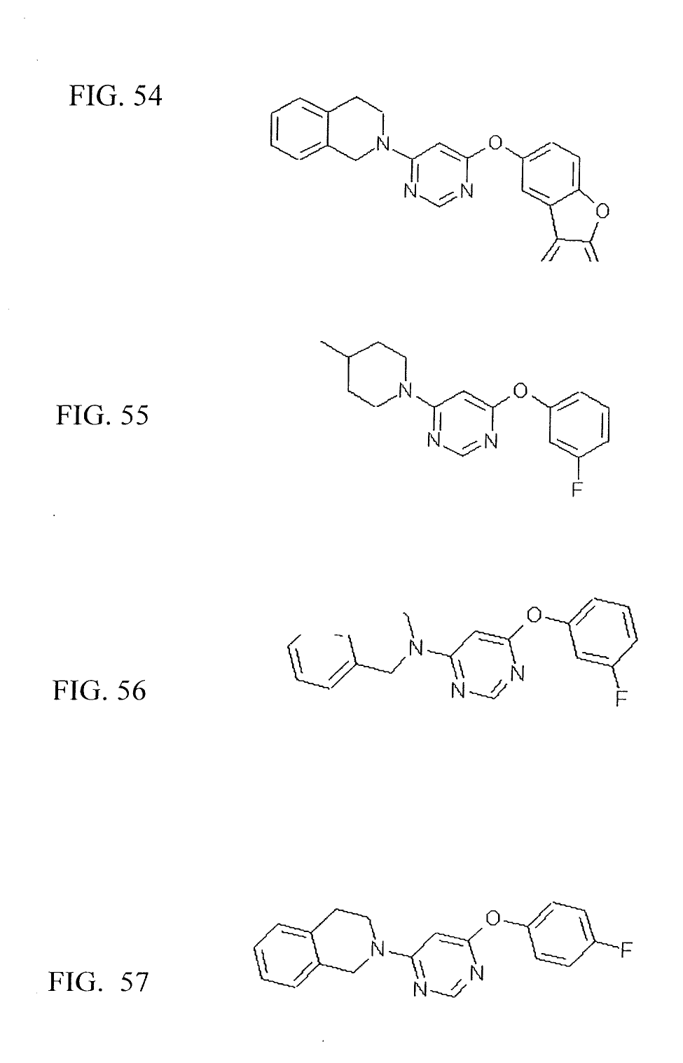

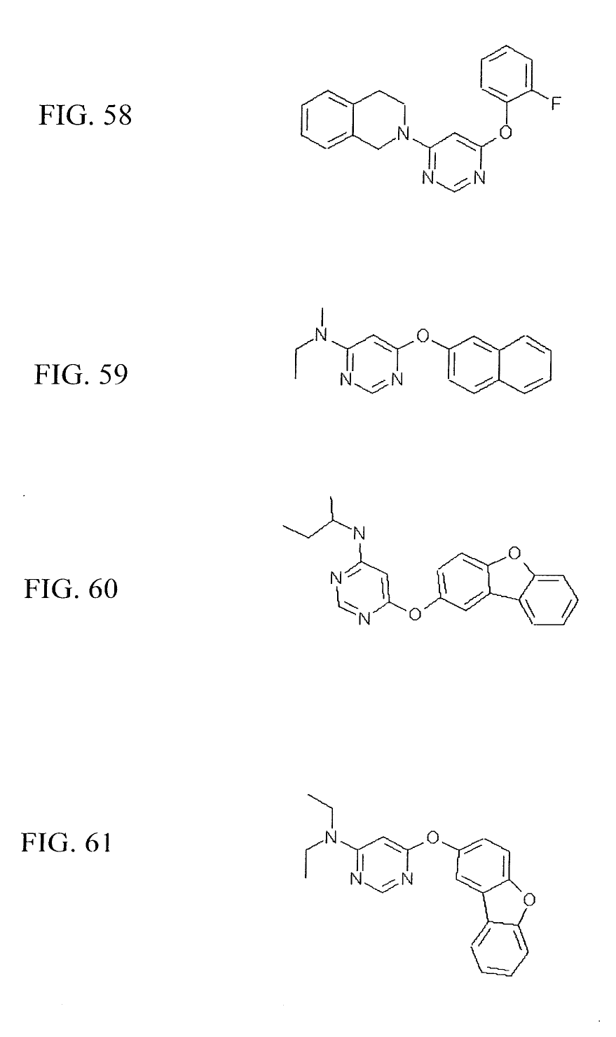

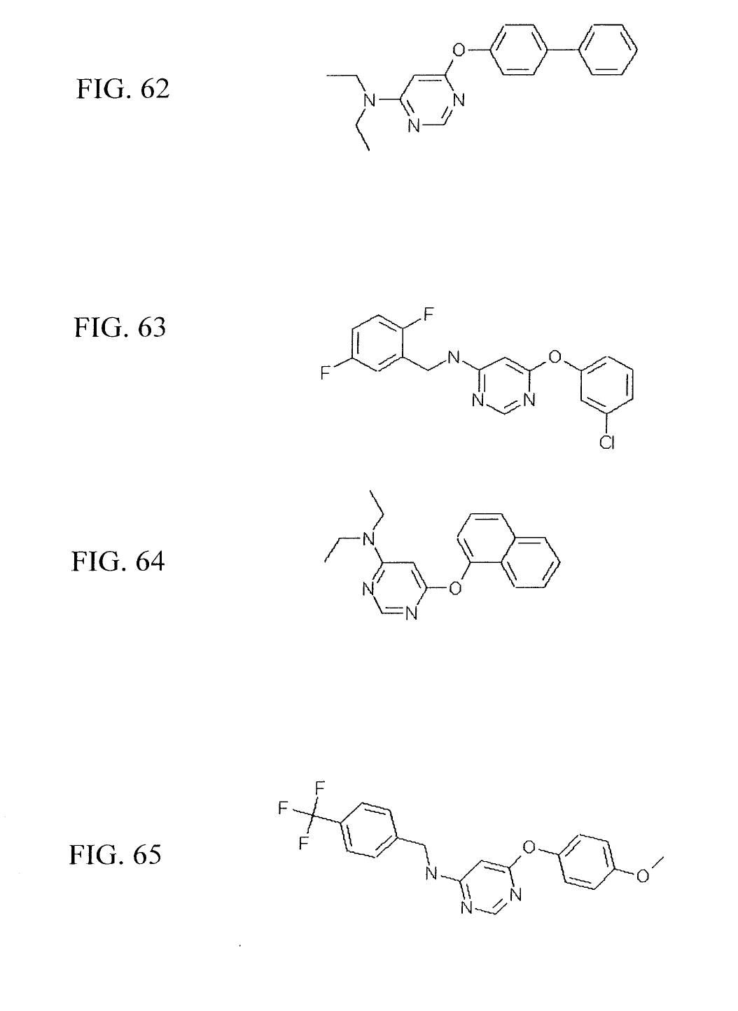

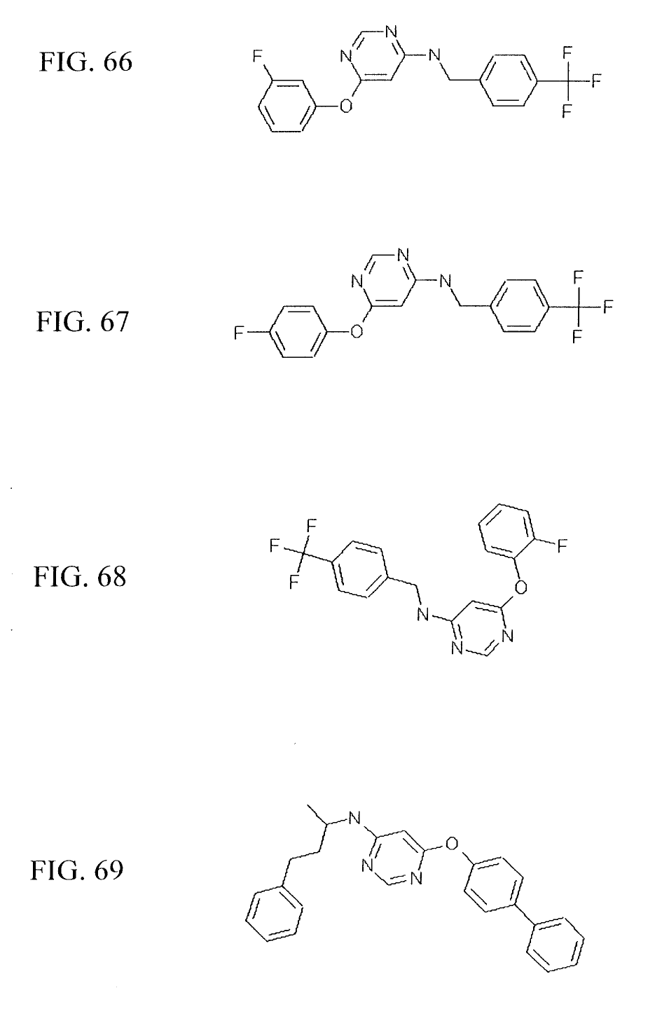

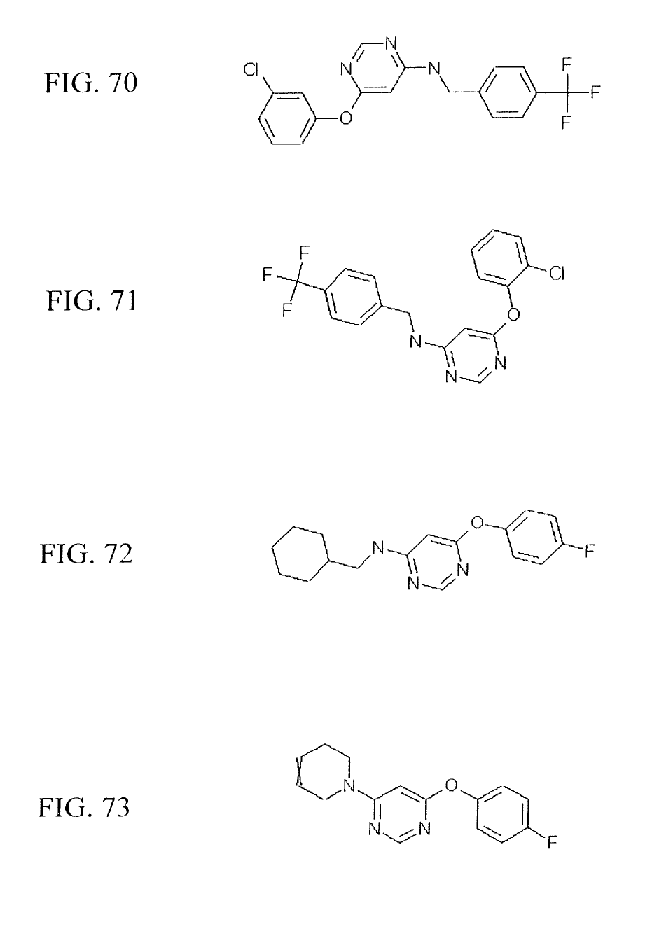

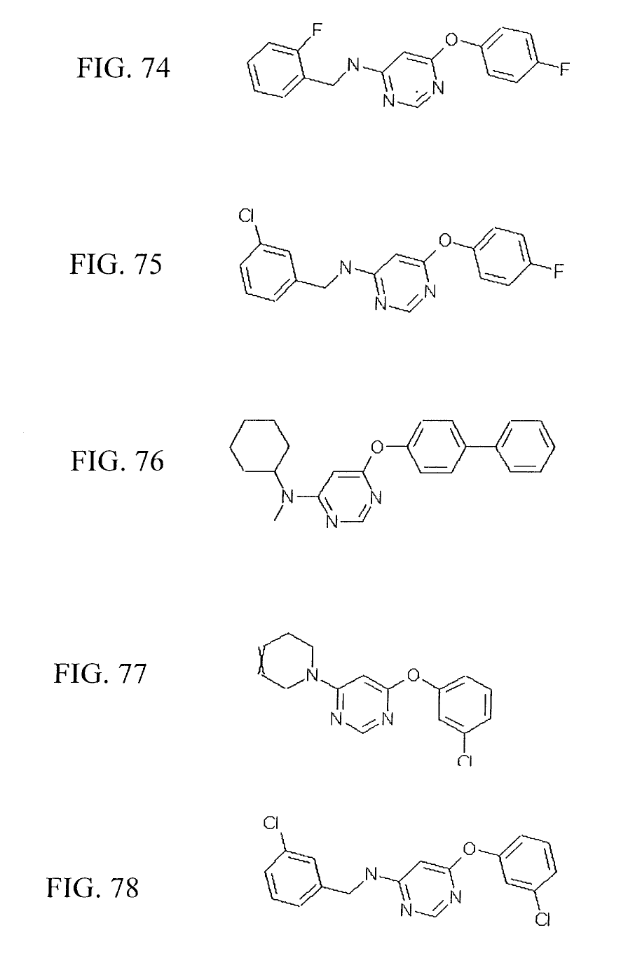

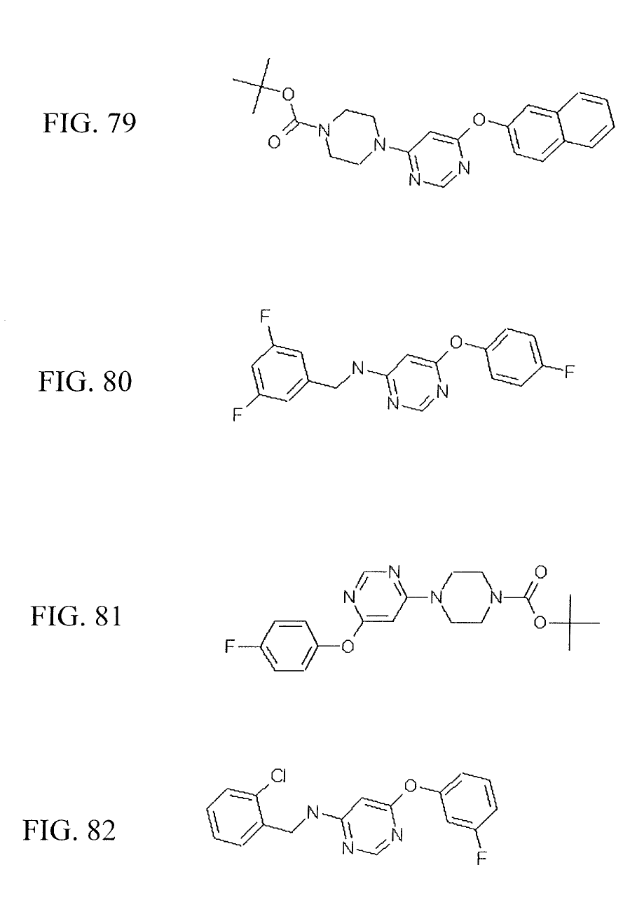

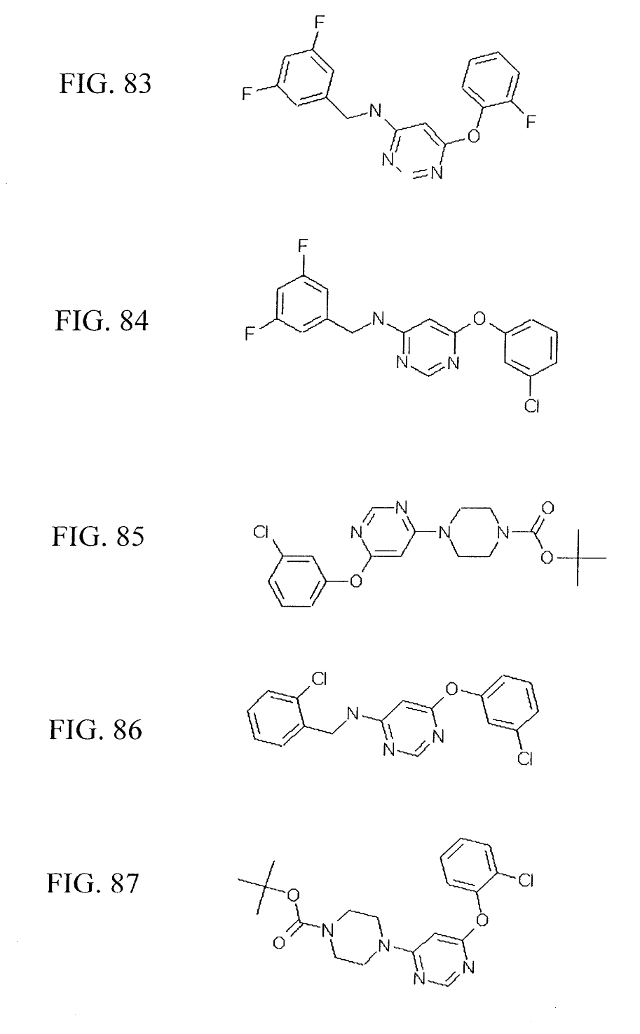

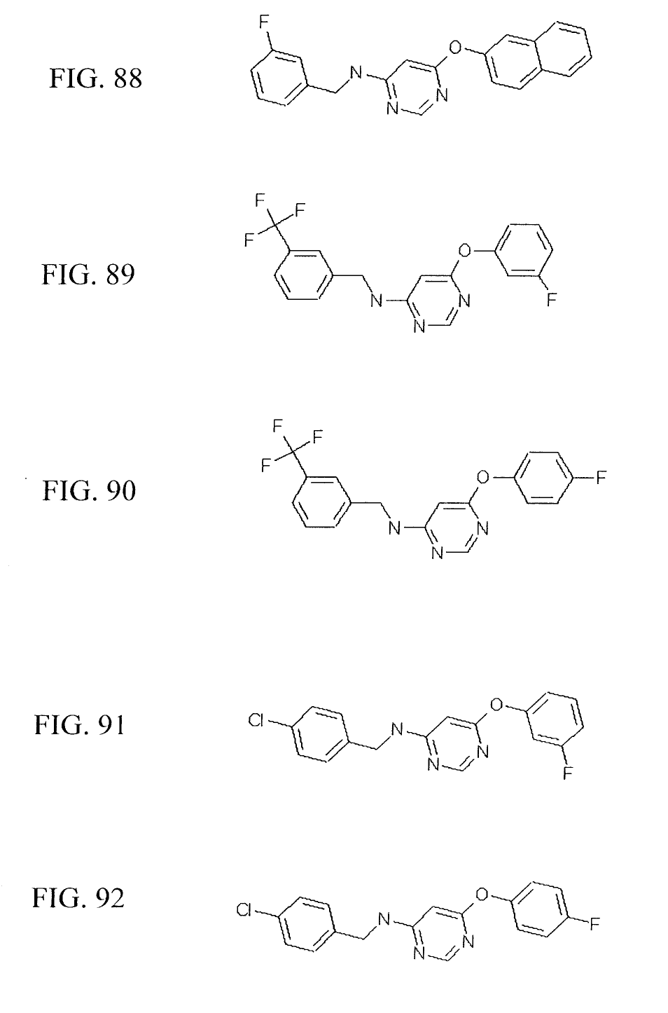

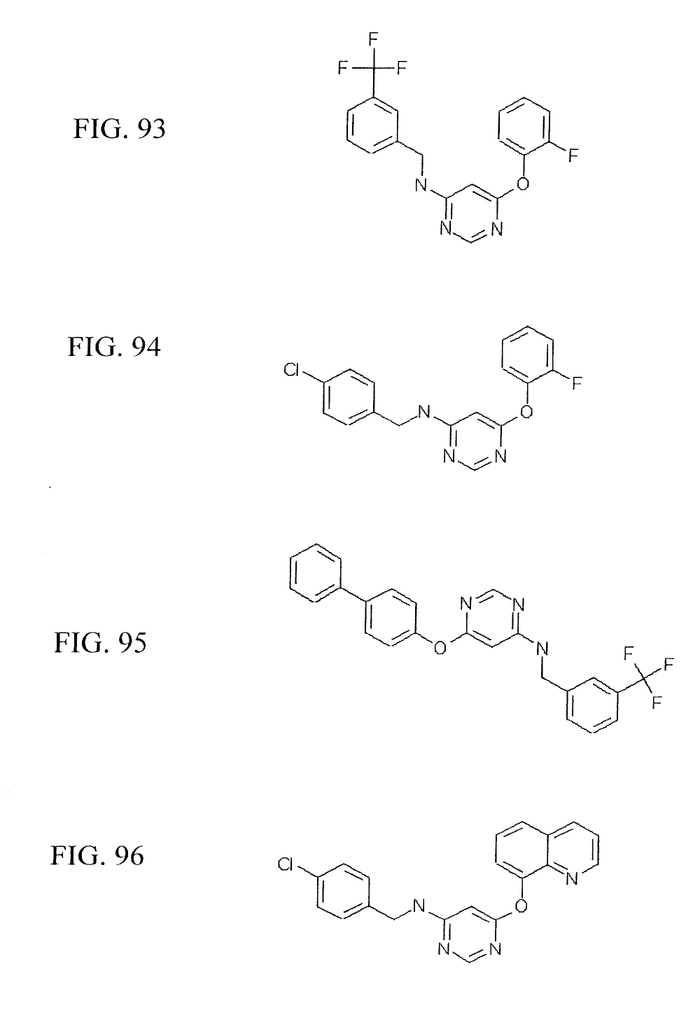

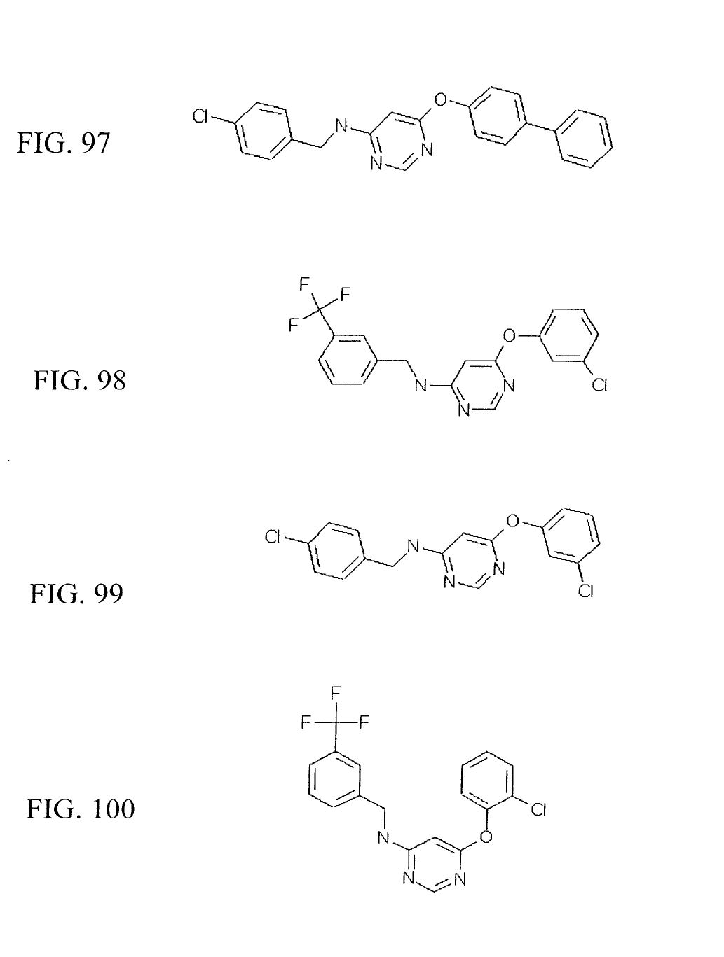

The process of the subject invention concerns the use of certain organic compounds to control the infestation of plants or animals by nematodes. These organic compounds comprise Formulae I, II, III, IV, and V, as well as structures 44, 45, and 46. In a particularly preferred embodiment of the subject invention, the anthelmintic compound is selected from the group consisting of Compounds 1-46 represented by Structures 1-46. Particularly preferred is the compound represented by Structure 32, and compounds related thereto as represented by Structure 47 depicted in FIG. 47, and as further exemplified by Structures 48-118 depicted in FIGS. 48 through 118. Preferred anthelmintic compounds useful in accord with the subject invention are represented by Structure 47, wherein

R₁ is H; C₁₋₅ alkyl; halogen; NOC₁₋₅; aryl (optionally substituted with halogen, phenyl, OC₁₋₅ alkyl, or halogenated C₁₋₅ alkyl); OC_{1-5;} COAr; or heterocycle (optionally substituted with C₁₋₅ alkyl);

R₂ is H; C₁₋₅ alkyl; halogen; NOC_{1-5;} aryl (optionally substituted with halogen, phenyl, OC₁₋₅ alkyl, or halogenated C₁₋₅ alkyl); OC₁₋₅; COAr; or heterocycle (optionally substituted with C₁₋₅ alkyl);

or R₁ and R₂ form an aryl, heteroaryl, or heterocycle which, optionally, is a component of a multicyclic group which further comprises an aryl group;

R₃ is H; aryl or heteroaryl (either of which is optionally substituted with halogenated C₁₋₅ alkyl, phenyl or halogen); C₃₋₁₂ cycloalkyl; or C₁₋₂₀ straight or branched alkyl which is optionally substituted with aryl (optionally substituted with halogenated alkyl or halogen);

R₄ is H; aryl or heteroaryl (either of which is optionally substituted with halogenated C₁₋₅ alkyl, phenyl or halogen); C₃₋₁₂ cycloalkyl; or C₁₋₂₀ straight or branched alkyl which is optionally substituted with aryl (optionally substituted with halogenated alkyl or halogen);

or R₃ and R₄ form a heterocycle which is optionally a component of a multicyclic group (which further comprises an aryl group) wherein said heterocycle is optionally substituted with C₁₋₅ alkyl, NOC, or COOC₁₋₅.

Generally, the anthelmintic compounds of the subject invention can be unsubstituted or substituted, saturated or unsaturated. The anthelmintic component of an anthelmintic composition used according to the subject invention may be a single anthelmintic compound or a mixture of two or more anthelmintic compounds. The subject compounds may be used in conjunction with other anthelmintic compounds, including the free acids and salts of the anthelmintic compounds of the present invention. The salts may be, for example, sodium or potassium salts, or ammonium salts. As would be apparent to the ordinary skilled artisan, physiologically acceptable acids and salts of the subject anthelmintic compounds can readily be made and used in accord with the teachings herein, and are hereby expressly included by reference to each compound or group of compounds. For example, “Compound AKC 340” or “Structure 48” is intended to include the physiologically acceptable acids and salts thereof.

Anthelmintic compounds specifically exemplified herein include Compounds 1-46 represented by Structures 1-46 above, and Compounds 48-118 represented by Structures 48-118 depicted in FIGS. 48-118.

The subject compounds used in the invention can be applied to animals, the living and feeding areas of animals, plants, or to the situs of plants needing nematode control. The anthelmintic compositions may be applied by, for example, drip and drench techniques. With the drip application, the subject compositions can be applied directly to the base of plants or to the soil root zone. The composition may be applied through already existing drip irrigation systems. This procedure is particularly applicable for ornamental plants, strawberries, tomatoes, potatoes, grapes, and vegetables. Alternatively, a drench application can be used. For treating plants, a sufficient quantity of the anthelmintic composition is applied such that the composition drains to the root area of the plants. An important aspect of the subject invention is the surprising discovery that certain 4-phenoxy-6-amino-pyrimidine compounds have excellent nematicidal activity at concentrations which are not phytotoxic.

The drench technique can be used for a variety of crops and for turf grasses. The drench technique can also be used for animals. Preferably, for administration to animals the anthelmintic composition would be administered orally to facilitate activity against internal nematode parasites. The compositions of the subject invention can readily be applied using the teachings provided herein.

In a preferred embodiment of the subject invention, an anthelmintic compound will be applied as an aqueous microemulsion. As described herein, the concentration of the active ingredient should be sufficient to control the nematode infestation without causing phytotoxicity to the desired plants. The concentration of anthelmintic compound may be, for example, from about 0.0001% to about 2%, preferably from about 0.025% to about 1%, and, most preferably, from about 0.05% to about 0.5%.

The anthelmintic composition used according to the subject invention can be applied in conjunction with one or more other nematicidal agents. The other nematicidal agent may, for example, be applied simultaneously or sequentially with the anthelmintic. Such other nematicidal agents include, for example, avermectins, the B.t.s, and fatty acids.

The avermectin compound used according to the subject invention may be any of the avermectins, milbemycins, or derivatives of either, having activity against nematodes. The avermectin's activity will be enhanced when combined with an anthelmintic compound as described herein. Thus, the specific combination of ingredients can be manipulated to provide the optimal composition for a particular application.

Standard concentrations of avermectins are well known to those skilled in the art. For example, the avermectin compounds can be employed in the combination of the subject invention at concentrations of from about 0.03 to about 110 parts per million (ppm). Preferably, from about 1 to about 5 ppm are employed.

As would be readily appreciated by a person skilled in the art, the delivery of the subject anthelmintic and/or avermectin compound can be calculated in terms of the active ingredient applied per unit area. For example, the subject anthelmintic may be applied at a rate of about 0.02 lb/acre to about 0.1 lb/acre and, preferably, from about 0.5 lb/acre to about 2 lbs/acre. Similarly, the avermectin product can be applied at a rate of up to about 16 oz. of formulated product (“AVID,” available from Merck) per acre. Preferably, about 4 oz. to about 8 oz. formulated “AVID” per acre would be used. Thus, the avermectin compound can be applied up to about 0.02 lb/acre. Preferably, the rate of avermectin is between about 0.005 lb/acre and 0.01 lb/acre. A person of ordinary skill in the art would readily appreciate that the desired application rate of the active ingredients could be achieved using a great variety of different concentrations of active ingredients while varying the application rate of the solution. Thus, a large quantity of dilute solution could be applied or a smaller quantity of a more concentrated solution.

A variety of different avermectins or related compounds can be used according to the subject invention. Ivermectin may also be used according to the subject invention, as may the milbemycins. For brevity, the term “avermectin” is used herein to refer to all the avermectins and their derivatives as well as related compounds such as the milbemycins and the ivermectins. “Derivatives” refer to chemical modifications of the avermectins or milbemycins which are well known and available to those skilled in this art. Such derivatives are described, for example, in U.S. Pat. No. 4,560,677. Avermectin is readily available under a variety of tradenames including “AVID,” “ZEPHYR,” “VERTIMEC,” and “AGRI-MEK.”

The anthelmintic compositions of the subject invention may also be used in conjunction with nematicidal agents other than the avermectins. For example, the anthelmintic compounds may be used with biological agents such as Bacillus thuringiensis or with nematicidal fungi. In this context, the anthelmintic composition could be applied at concentrations which would not antagonize the action of the biological agent. The biologically active agent may be in a live proliferative form or may be in a dead stabilized form as described, for example, in U.S. Pat. Nos. 4,695,462 and 4,695,455. Furthermore, the anthelmintic compositions of the subject invention may be used with plants which are specifically bred or engineered for nematode resistance. The plants may, for example, be transformed with B.t. genes which confer nematode resistance or may simply be hybrids or varieties selected for such resistance. The anthelmintic compositions of the subject invention are particularly effective against free- living ectoparasitic nematodes and, therefore, combined use with plants selected for endoparasitic nematode resistance is highly advantageous.

The subject invention further relates to the surprising discovery that the anthelmintics of the subject invention have ovicidal activity against nematode eggs. Thus, in another embodiment, provided are methods for killing the eggs of nematodes, including those within cysts or egg masses that are commonly formed by Heterodera, Globodera, and Meloidogyne (cyst and root-knot) species.

The ovicidal compositions according to the subject invention are particularly useful for preplant applications in nematode-control schemes. In addition, the ovicidal compositions of the subject invention can be advantageously used as postplant nematicides, especially because of their relatively low phytotoxicity. In the latter embodiments, ovicidal compositions of the subject invention can be delivered, after planting and at appropriate, essentially non-phytotoxic concentrations of anthelmintic compounds, along with irrigation water and/or plant nutrients to ensure a continuous zone of nematode protection to the enlarging plant root mass. Thus, when applied using these techniques, which include drench or drip systems as are known in the art, phytopathogenic nematodes in their vermiform (wormlike) and egg stages are controlled.

Anthelmintic compounds having Formulae I, II, III, IV, and V, Structure 47 and most preferably structures 1-46, and particularly Structure 32 and Structures 48-118, are used in preferred embodiments for killing nematode eggs. In addition, microemulsions of the subject compounds are highly preferred for ovicidal applications. In preferred embodiments, the anthelmintic compound(s) will be present in a concentration of greater than about 150 ppm. More preferably, the concentration will be greater than about 200 ppm; most preferably it will be about 250 ppm or more. For certain conditions, the anthelmintic compounds should be applied at high concentrations of about 1,000 ppm to about 5,000 ppm or more.

In light of the subject disclosure, one skilled in the art could readily, use a variety of application techniques and formulations to prevent the hatching of nematode eggs in a variety of agricultural, farm-related, and garden-related settings.

Examples of animal parasitic nematodes against which the subject compounds can be used include the following:

Amblyomma spp.

Babesia spp. (RBC)

Bunostomum spp.

Calliphorid larvae

Capillaria spp.

Chabertia ovina

Chorioptes

Cooperia spp.

Cryptosporidium sp.

Damalinia ovis

Damalinia caprae

Demodex

Dermacentor spp.

Dicrocoelium dentriticum

Dictyocaulus filaria

Echinococcus hydatid cyst

Eimeria spp.

Elaeophora schneideri

Fasciola hepatica

Fasciola gigantica

Fascioloides magna

Giardia sp.

Gongylonema spp.

Haematobia irritans

Haemonchus contortus contortus

Ixodes

Linguatula serrata larvae

Linguatula serrata nymphs

Linognathus spp.

M. domestica

Marshallagia marshalli

Melophagus ovinus

Moniezia benedeni

Moniezia expansa

Muellerius capillaris

Musca autumnalis

Nematodirus spp.

Oesophagostomum spp.

Oestrus ovis

Ornithodoros

Ostertagia circumcincta

Ostertagia trifurcata

Otobius

Paramphistomum sp.

Parelaphostrongylus tenuis

Protostrongylus sp.

Psoroptes

Rhipicephalus spp.

Sarcoptes scabiei

Sarcocystis spp.

Sarcocystis spp. cysts

Schistosoma spp.

Stomoxys calcitrans

Strongyloides papillosus

Taenia hydatigena cysticerci

Taenia multiceps coenurus

Taenia ovis cysticerci

Thelazia

Thysanosoma actinoides

Theileria spp.C)

Toxocara vitulorum

Toxoplasma gondii

Toxoplasma gondii cysts

Trichostrongylus axei

Trichostrongylus spp.

Trichuris ovis

Trypanosoma spp. (plasma)

It has been found that helminth, acarid and arthropod endo- and ectoparasitic infestations may be controlled, prevented or eliminated, by applying to, injecting or orally dosing said animals with an endo- or ectoparasiticidally effective amount of the subject anthelmintic compounds, preferably the above-described structure 1-46 compounds. This may be achieved by applying the compound to the skin, hide and/or hair of the animals, or injecting or orally dosing said animals with a solid or liquid formulated composition.

For control of flea infestations, treatment of the infested animal to control adults in conjunction with treatment of the area occupied by the infested animal to control flea larvae is recommended. The compositions of the present invention may be admixed with suitable carriers for application to interior and/or exterior areas for control of flea larvae.

The compositions of the present invention may be employed as animal feeds, animal feed premixes or feed concentrates. Feed concentrates and feed premixes, useful in the practice of the invention, may be prepared by admixing about 0.25% to 35% by weight of a subject anthelmintic compound, preferably a structure 1-46 compound, with about 99.75% to 65% by weight of a suitable agronomic carrier or diluent. Carriers suitable for use include 0.75% to 35% by weight of a physiologically acceptable alcohol such as benzyl alcohol, phenethyl alcohol or propylene glycol, 0 to about 10% by weight of a vegetable oil such as corn oil or soybean oil, or propylene glycol and about 30% to 95% by weight of a sorptive, edible organic carrier such as corn grits, wheat middlings, soybean meal, expanded corn grits, extracted corn meal or the like or a sorptive silica or a silicate. These feed premixes or concentrates may be admixed with the appropriate amount of animal feed to provide the animals with about 0.5 ppm to 1000 ppm and preferably about 1 ppm to 500 ppm of the compound in the animal's diet. These premixes or concentrates may also be used as top dressings for the animal's daily ration and applied across the top of the daily ration in sufficient amount to provide the animal with about 0.5 ppm to 1,000 ppm and preferably about 1 ppm to 500 ppm of the active ingredient, based on the animal's total feed.

The subject anthelmintic compounds, and particularly the structure 1-46 compounds, most particularly Structure 32 and Structures 48-118 compounds, may be administered to the animals in or with their drinking water.

The compound may also be administered in the form of a pill, tablet, bolus, implant, capsule, or drench, containing sufficient anthelmintic compound to provide the treated animal with about 0.01 mg/kg to 100 mg/kg of animal body weight per day of the compound. These dosage forms are prepared by intimately and uniformly mixing the active ingredient with suitable finely divided diluents, fillers, disintegrating agents and/or builders such as starch, lactose, talc, magnesium stearate, vegetable gums, or the like. These unit dosage formulations may be varied with respect to the total weight and content of anthelmintic compound depending upon the kind and size of the animal to be treated, the severity or type of infection encountered and the weight of the host.

Alternatively, the anthelmintic compound may be administered to animals parenterally, for example, by intraruminal, intramuscular, or subcutaneous injection in which the active ingredient is dissolved or dispersed in a liquid carrier. For this type administration the compound may be dispersed in a physiologically acceptable solvent for subcutaneous injection, or it may be dispersed in a fat or wax or mixture thereof containing an oil, buffer, surfactant, stabilizer, preservative and salt. Components useful in these preparations include carbowax, aluminum monostearate gel, diethyl succinate, soya oil, glyceral dioleate, saline, and capric/caprylic triglycerides.

The subject anthelmintic compounds may also be applied topically to the larger animals such as swine, sheep, cattle, and horses and companion animals such as dogs and cats in the form of aqueous dips or sprays. For this type administration, the active compound is generally prepared as a wettable powder, emulsifiable concentrate, aqueous flowable, or the like, which is mixed with water at the site of treatment and applied topically to the hide, skin, or hair of the animal. Such sprays or dips usually contain about 0.5 ppm to 5,000 ppm and preferably about 1 ppm to 3,000 ppm of the compound.

Advantageously, the subject anthelmintic compounds may also be prepared as pour-on formulations and poured on the backs of the animals such as swine, cattle, sheep, horses, poultry, and companion animals to protect them against infestation by nematodes, acarids, and arthropod endo- and ectoparasites. Such pour-on compositions are generally prepared by dissolving, dispersing, or emulsifying the anthelmintic compound in a suitable nontoxic pharmacologically acceptable diluent for pour-on and administration. The diluent must be compatible with the compound and should not be a source of irritation or damage to the animals hide, skin, or hair. Such diluents include vegetable oils, spreading oils, polyhydric alcohols, aliphatic or aromatic hydrocarbons, esters of fatty acids, and lower alkyl ketones.

A typical pour-on formulation includes about 0.5% to 30% by weight of the anthelmintic compound, about 30% to 60% by weight of an aliphatic or aromatic hydrocarbon, mono or polyhydric alcohol, lower alkyl ketone or mixtures thereof, 0 to about 20% by weight of a vegetable or mineral oil and about 0.5% to 30% by weight of a spreading oil. Another typical pour-on contains about 45% by weight of xylene, about 15% by weight of the anthelmintic compound, about 10% by weight of corn oil or mineral oil, about 25% by weight of cyclohexanone and about 5% by weight of other pharmacologically acceptable spreading agents, antifoam agents, surfactants, or the like.

The subject anthelmintic compounds may also be prepared as ear tags for animals, particularly quadrupeds such as cattle and sheep. The tags may be prepared by stirring together about 55% to 60% by weight of a vinyl dispersion resin, having an inherent viscosity of about 1.20 and an average particle size of about 0.75 microns, a curing temperature range of about 120° C. to 180° C., with about 28% by weight of butylbenzylphthalate. Stirring is continued, and about 1.5% by weight of ca/Zn stearate stabilizer is added along with about 7.0% of the compound and 2.8% of epoxidized soybean oil. The resulting mixture is deaerated for 15 to 20 minutes at 125 mm/Hg. This mixture can be coated on an ear tag blank by dipping and the resulting tag cured at about 145° C. to 150° C. for about five minutes.

The compounds of Formulae I-V, Structure 47, particularly Structures 1-46, and particularly Structures 32 and 48-118 are nematicidal and can be used to control nematodes in crop plants. Therefore, in a further preferred aspect of the invention, there is provided a method for killing or controlling nematodes which comprises applying to the locus of the pests or to a plant susceptible to attack by the pest an effective amount of a compound having any of Structures 1-46, preferably Structure 47, and particularly Structures 32 and 48-118, as defined herein.

The term “controlling” extends to non-lethal effects which result in the reduction or prevention of damage to the host plant or animal and the limitation of nematode population increase. These effects may be the result of chemical induced disorientation, immobilisation, or hatch prevention or induction. The chemical treatment may also have deleterious effects on nematode development, reproduction, or viability.

The compounds of the invention can be used against both plant-parasitic nematodes and nematodes living freely in the soil. Examples of plant-parasitic nematodes are: ectoparasites, for example Xiphinema spp., Longidorus spp., and Trichodorous spp.; semi-endoparasites, for example, Tylenchulus spp.; migratory endoparasites, for example, Pratylenchus spp., Radopholus spp., and Scutellonema spp.; sedentary endoparasites, for example, Heterodera spp., Globodera spp., and Meloidogyne spp.; and stem and leaf endoparasites, for example, Ditylenchus spp., Aphelenchoides spp., and Hirshmaniella spp.

The Formulae I-V compounds, Structure 47 compounds, and preferably the compounds of Structures 1-46, more preferably the compounds of Structures 32 and 48-118, display nematicidal activity against different types of nematodes including the cyst nematode. The subject compounds may also be used to combat and control infestations of insect pests such as Lepidoptera, Diptera, Homoptera, and Coleoptera (including Diabrotica i.e. corn rootworms) and also other invertebrate pests, for example, acarine pests. The insect and acarine pests which may be combated and controlled by the use of the invention compounds include those pests associated with agriculture (which term includes the growing of crops for food and fiber products), horticulture and animal husbandry, forestry, the storage of products of vegetable origin, such as fruit, grain and timber, and also those pests associated with the transmission of diseases of man and animals. Examples of insect and acarine pest species which may be controlled by the subject compounds include:

Myzus persicae (aphid)

Aphis gossypii (aphid)

Aphis fabae (aphid)

Megoura viceae (aphid)

Aedes aegypti (mosquito)

Anopheles spp. (mosquitos)

Culex spp. (mosquitos)

Dysdercus fasciatus (capsid)

Musca domestica (housefly)

Pieris brassicae (white butterfly)

Plutella maculipennis (diamond back moth)

Phaedon cochleariae (mustard beetle)

Aonidiella spp. (scale insects)

Trialeuroides spp. (white flies)

Bemisia tabaci (white fly)

Blattella germanica (cockroach)

Periplaneta americana (cockroach)

Blatta orientalis (cockroach)

Spodoptera littoralis (cotton leafworm)

Hellothis virescens (tobacco budworm)

Chortiocetes terminifera (locust)

Diabrotica spp. (rootworms)

Agrotis spp. (cutworms)

Chilo partellus (maize stem borer)

Nilaparvata lugens (planthopper)

Nephotettix cincticeps (leafhopper)

Panonychus ulmi (European red mite)

Panonychus citri (citrus red mite)

Tetranychus urticae (two-spotted spider mite)

Tetranychus cinnabarinus (carmine spider mite)

Phyllcoptruta oleivora (citrus rust mite)

Polyphagotarsonemus latus (broad mite)

Brevipalpus spp. (mites)

In order to apply the compound to the locus of the nematode, insect, or acarid pest, or to a plant susceptible to attack by the nematode, insect, or acarid pest, the compound is usually formulated into a composition which includes in addition to at least one of the subject anthelmintic compounds suitable inert diluent or carrier materials, and/or surface active agents. Thus, in two further aspects of the invention there is provided a nematicidal, insecticidal, or acaricidal composition comprising an effective amount of a subject anthelmintic compound and preferably of any of structures 1-46, preferably compounds of Structure 47, more preferably as exemplified by Structures 32 and 48-118, as defined herein and an inert diluent or carrier material and optionally a surface active agent.

The amount of active ingredient generally applied for the control of nematode pests is from 0.01 to 10 kg per hectare, and preferably from 0.1 to 6 kg per hectare.

The compositions containing the active ingredient can be applied to the soil, plant or seed, to the locus of the pests, or to the habitat of the pests, in the form of dusting powders, wettable powders, granules (slow or fast release), emulsion or suspension concentrates, liquid solutions, emulsions, seed dressings, fogging/smoke formulations or controlled release compositions, such as microencapsulated granules or suspensions.

Dusting powders are formulated by mixing the active ingredient with one or more finely divided solid carriers and/or diluents, for example natural clays, kaolin, pyrophyllite, bentonire, alumina, montmorillonite, kieselguhr, chalk, diatomaceous earths, calcium phosphates, calcium and magnesium carbonates, sulphur, lime, flours, talc, and other organic and inorganic solid carriers.

Granules are formed either by absorbing the active ingredient in a porous granular material for example pumice, attapulgite clays, fullers earth, kieselguhr, diatomaceous earths, ground corn cobs, and the like, or on to hard core materials such as sands, silicates, mineral carbonates, sulphates, phosphates, or the like. Agents which are commonly used to aid in impregnation, binding or coating the solid carriers include aliphatic and aromatic petroleum solvents, alcohols, polyvinyl acetates, polyvinyl alcohols, ethers, ketones, esters, dextrins, sugars, and vegetable oils with the active ingredient. Other additives may also be included, such as emulsifying agents, wetting agents, or dispersing agents.

Microencapsulated formulations (microcapsule suspensions CS) or other controlled release formulations may also be used, particularly for slow release over a period of time, and for seed treatment.

Alternatively the compositions may be in the form of liquid preparations to be used as dips, irrigation additives or sprays, which are generally aqueous dispersions or emulsions of the active ingredient in the presence of one or more known wetting agents, dispersing agents or emulsifying agents (surface active agents). The compositions which are to be used in the form of aqueous dispersions or emulsions are generally supplied in the form of an emulsifiable concentrate (EC) or a suspension concentrate (SC) containing a high proportion of the active ingredient or ingredients. An EC is a homogeneous liquid composition, usually containing the active ingredient dissolved in a substantially non-volatile organic solvent. An SC is a fine particle size dispersion of solid active ingredient in water. To apply the concentrates they are diluted in water and are usually applied by means of a spray to the area to be treated. For agricultural or horticultural purposes, an aqueous preparation containing between 0.0001% and 0.1% by weight of the active ingredient (approximately equivalent to from 5-2000 g/ha) is particularly useful.

Suitable liquid solvents for ECs include methyl ketone, methyl isobutyl ketone, cyclohexanone, xylenes, toluene, chlorobenzene, paraffins, kerosene, white oil, alcohols, (for example, butanol), methylnaphthalene, trimethylbenzene, trichloroethylene, N-methyl-2-pyrrolidone, and tetrahydrofurfuryl alcohol (THFA).

Wetting agents, dispersing agents, and emulsifying agents may be of the cationic, anionic, or non-ionic type. Suitable agents of the cationic type include, for example, quaternary ammonium compounds, for example cetyltrimethyl ammonium bromide. Suitable agents of the anionic type include, for example, soaps; salts of aliphatic monoesters of sulphuric acid, for example sodium lauryl sulphate; salts of sulphonated aromatic compounds, for example sodium dodecylbenzenesulphonate; sodium, calcium or ammonium lignosulphonate; or butylnaphthalene sulphonate; and a mixture of the sodium salts of diisopropyl- and triisopropylnaphthalene sulphonates. Suitable agents of the non-ionic type include, for example, the condensation products of ethylene oxide with fatty alcohols such as oleyl alcohol or cetyl alcohol; or with alkyl phenols such as octyl phenol, nonyl phenol, and octyl cresol. Other non-ionic agents are the partial esters derived from long chain fatty acids and hexitol anhydrides, the condensation products of the said partial esters with ethylene oxide, and the lecithins.

These concentrates are often required to withstand storage for prolonged periods and after such storage, to be capable of dilution with water to form aqueous preparations which remain homogeneous for a sufficient time to enable them to be applied by conventional spray equipment. The concentrates may preferably contain 1-85% by weight of the active ingredient or ingredients. When diluted to form aqueous preparations such preparations may contain varying amounts of the active ingredient depending upon the purpose for which they are to be used.

The subject anthelmintic compounds may also be formulated as powders (dry seed treatment DS or water disperible powder WS) or liquids (flowable concentrate FS, liquid seed treatment LS), or microcapsule suspensions CS for use in seed treatments. The formulations can be applied to the seed by standard techniques and through conventional seed treaters. In use the compositions are applied to the nematodes, to the locus of the nematodes, to the habitat of the nematodes, or to growing plants liable to infestation by the nematodes, by any of the known means of applying pesticidal compositions, for example, by dusting, spraying, or incorporation of granules.

The compounds of the invention may be the sole active ingredient of the composition or they may be admixed with one or more additional active ingredients such as nematicides, agents which modify the behavior of nematodes (such as hatching factors), insecticides, synergists, herbicides, fungicides or plant growth regulators where appropriate.

Suitable additional active ingredients for inclusion in admixture with the compounds of the invention may be compounds which will broaden the spectrum of activity of the compounds of the invention or increase their persistence in the location of the pest. They may synergise the activity of the compound of the invention or complement the activity for example by increasing the speed of effect or overcoming repellency. Additionally multi-component mixtures of this type may help to overcome or prevent the development of resistance to individual components.

The particular additional active ingredient included will depend upon the intended utility of the mixture and the type of complementary action required. Examples of suitable insecticides include the following:

a) Pyrethroids such as permethrin, esfenvalerate, deltamethrin, cyhalothrin in particular lambda-cyhalothrin, biphenthrin, fenpropathrin, cyfluthrin, tefluthrin, fish safe pyrethroids for example ethofenprox, natural pyrethrin, tetramethrin, s-bioallethrin, fenfluthrin, prallethrin, and 5-benzyl-3-furylmethyl-(E)-(1R,3 S)-2,2-dimethyl-3-(2-oxothiolan-3-ylidenemethyl) cyclopropane carboxylate;

b) Organophosphates such as profenofos, sulprofos, methyl parathion, azinphos-methyl, demeton-s-methyl, heptenophos, thiometon, fenamiphos, monocrotophos, profenophos, triazophos, methamidophos, dimethoate, phosphamidon, malathion, chloropyrifos, phosalone, terbufos, fensulphothion, fonofos, phorate, phoxim, pyrimiphos-methyl, pyrimiphos-ethyl, fenitrothion, or diazinon;

c) Carbamates (including aryl carbamates) such as pirimicarb, cloethocarb, carbofuran, furathiocarb, ethiofencarb, aldicarb, thiofurox, carbosulphan, bendiocarb, fenobucarb, propoxur, or oxamyl;

d) Benzoyl ureas such as triflumuron or chlorofluazuron;

e) Organic tin compounds such as cyhexatin, fenbutatin oxide, or azocyclotin;

f) Macrolides such as avermectins or milbemycins, for example such as abamectin, avermectin, and milbemycin;

g) Hormones and pheromones;

h) Organochlorine compounds such as benzene hexachloride, DDT, endosulphan, chlordane, or dieldrin;

i) Amidines, such as chlordimeform or amitraz;

j) Fumigant agents;

k) nitromethylenes such as imidacloprid.

In addition to the major chemical classes of insecticide listed above, other insecticides having particular targets may be employed in the mixture if appropriate for the intended utility of the mixture. For instance, selective insecticides for particular crops, for example stemborer specific insecticides for use in rice such as cartap or buprofezin, can be employed. Alternatively, insecticides specific for particular insect species/stages, for example, ovo-larvicides such as chlofentezine, flubenzimine, hexythiazox, and tetradifon; motilicides such as dicofol or propargite; acaricides such as bromopropylate or chlorobenzilate; or growth regulators such as hydramethylon, cyromazin, methoprene, chlorfluazuron, and diflubenzuron may also be included in the compositions.

Examples of suitable synergists for use in the compositions include piperonyl butoxide, sesamax, safroxan, and dodecyl imidazole.

Suitable herbicides, fungicides, and plant-growth regulators for inclusion in the compositions will depend upon the intended target and the effect required.

An example of a rice selective herbicides which can be included is propanil, an example of a plant growth regulator for use in cotton is “Pix”, and examples of fungicides for use in rice include blasticides such as blasticidin-S. The ratio of the compound of the invention to the other active ingredient in the composition will depend upon a number of factors including type of target, effect required from the mixture, etc. However in general, the additional active ingredient of the composition will be applied at about the rate as it is usually employed, or at a slightly lower rate if synergism occurs.

The anthelmintic compounds according to the invention also show fungicidal activity and may be used to control one or more of a variety of plant pathogens. In a further aspect the invention therefore includes a method of combating fungi which comprises applying to a plant, to a seed of a plant, or to the locus of the plant or seed a fungicidally effective amount of a compound as herein defined or a composition containing the same. The invention further includes a fungicidal composition comprising a fungicidally effective amount of a compound as herein defined and a fungicidally acceptable carrier or diluent therefor.

Examples of plant pathogens which the compounds or fungicidal compositions of the invention may control, methods by which fungi may be combatted and the form of suitable compositions, including acceptable carriers and diluents; adjuvants such as wetting, dispersing, emulsifying, and suspending agents; and other ingredients, such as fertilisers and other biologically active materials, are described, for instance, in International application No. WO 93/08180, the content of which is incorporated herein by reference.

All of the U.S. patents cited herein are hereby incorporated by reference.

Following are examples which illustrate procedures for practicing the invention. These examples should not be construed as limiting. All percentages are by weight and all solvent mixture proportions are by volume unless otherwise noted. For clarity the following abbreviations shall be used throughout the examples:

| | |
|---|---|
| ACD: | Available Chemicals Directory |
| AM: | Amine |
| DCE: | Dichloroethane |
| DMSO: | Dimethylsulfoxide |
| TEA: | Triethylamine |
| TLC: | Thin Layer Chromatography |
| dH₂O: | Distilled Water |

### EXAMPLE 1

### Preparation of Anthelmintic Compounds 1-46

The anthelmintic compounds of the subject invention can readily be produced using procedures well known to those skilled in the art.

A variety of anthelmintic compounds useful according to the subject invention can be readily prepared by a person skilled in this art having the benefit of the subject disclosure.

### EXAMPLE 2

### Nematicidal Activity of Anthelmintic Compositions 1-31

Caenorhabditis elegans adults were grown on Nematode Growth Medium (NGM) until they produced eggs, then the adults were removed.

The eggs were allowed to hatch, and the L1 larvae collected. See The Neniatode Caenorhabditis elegans (1988) Cold Spring Harbor Laboratory Press. Using a Matrix Programmable Pipette, the L1s were distributed into 96-well tissue culture plates, 20 L1 in 50 μl NGM per well. Antibiotic/Antimyotic was added to each well, and 1% by weight E. coli strain HB101. The subject anthelmintic compounds were stored at 5 mM in 100% DMSO. 0.7 μl of compounds 1-31 were added to the left-most column of wells to yield a final concentration of 70 μM in 1.4% DMSO, with 1.4% DMSO only as the control. The compounds were then subjected to 5 more 3-fold dilutions from left to right to yield 6 column concentrations of 70 μM, 23.3 μM, 7.8 μM, 2.6 μM, 0.9 μM, and 0.3 μM. Plates were stored in air-tight Rubbermaid plastic boxes at 20° C. The nematodes had cleared all control wells by day 4, and nematode viability was scored by visual examination under a 100× dissecting microscope on day 5. A visual viability scoring system was used as follows:

### WORM VISUAL SCORING GUIDE

Lethality:

| | |
|---|---|
| Dead | only stiff L1s (no movement) |
| Dead (L4) | worms are dead, but at a later larval stage |
| L1 | majority of worms are L1 (based on size) |
| | worms move when plate is tapped |
| L2 | majority of worms are L2 (based on size) |
| L3 | majority of worms are L3 (based on size) |
| L4 | majority of worms are L4 (based on size) |

Partial Penetrance:

| | |
|---|---|
| AD | majority of worms are adult |
| #AD | 5 adult worms or less |

Broodsize Reductions:

| | | |
|---|---|---|
| B! | sterile | (0-25 progeny) |
| B | low broodsize | (25-100 progeny) |
| ˜B | moderate broodsize | (100-250 progeny) |
| < | reduced broodsize | (250-500 progeny) |
| OK | no effect | (˜1000+ progeny) |

If several classes of worms exist in a well, then all classes are scored. If adults are present, then the brood score is also recorded. Thus, “L1/L2” would mean a mixture of L1's and L2's are present in the well. “L4/#AD/B” would mean that a mixture of L4's and adults are resent in the well. The “#AD” would mean that there are 6 or less adults, and the “B” would mean that there were 100 progeny or less.

The results are reported in Table 1. Column VI has a compound concentration of 70 μM with sequential 3-fold dilutions reported in columns V2, V3, V4, V5, and V6, respectively, such that the V6 concentration was 0.3 μM.

TABLE 1

| Dose Response Tracking | | | | | | | | | | |
|---|---|---|---|---|---|---|---|---|---|---|
| | HTS Tracking | Structure | | Well | 5 Day Visual Score | | | | | |
| DR# | Library # | # | Source P | Address | V1 | V2 | V3 | V4 | V5 | V6 |
| 1575 | AKC 111 | 1 | N2#93 | 5081:D10 | Dead | Dead | L2 | Dead(L3/L | #AD/B | OK |
| 1647 | AKC 112 | 2 | N2#98 | 5090:A10 | L2/L3 | L2/L3 | L2/L3 | L3/L4 | #AD/˜B | OK |
| 1466 | AKC 113 | 3 | N2#85 | 5061:A10 | Dead | Dead | Dead | Dead(L2/L | L2/Dead(A | L4/#AD/B |
| 1469 | AKC 107 | 4 | N2#86 | 5061:D10 | Dead | Dead(L2/L3) | L2/L3 | Dead(L4) | L4 Dead(A | L2/Dead(L |
| 1477 | AKC 114 | 5 | N2#86 | 5061:D11 | Dead | L3/Dead(L4) | L3 | L3/Dead(L | L2/L3 | B |
| 1476 | AKC 108 | 6 | N2#86 | 5061:C11 | Dead | Dead | L1 | L1 | L1/L2 | L1/L3 |
| 1473 | AKC 115 | 7 | N2#86 | 5061:H10 | Dead | Dead(L2/L3) | L2/Dead(L2) | Dead(L2) | Dead(L2/L | L2/L3 |
| 035 | AKC 119 | 11 | N2#126 | 5393:B4 | #AD/B | #AD/B | #AB/B | #AD/B | #AD/B | < |
| 2059 | AKC 110 | 12 | N2#128 | 5399:C4 | L1 | L1 | L1 | L1 | L1 | L1 |
| 2083 | AKC 120 | 13 | N2#130 | 5449:C4 | L1 | L2/L3 | L1 | L1/L2 | #AD/˜B | OK |
| 2032 | AKC 121 | 14 | N2#126 | 5389:C4 | L1 | L1 | L1/L2 | #AD/˜B | #AD/B | < |
| 2029 | AKC 2153 | 15 | N2#126 | 5379:C4 | L1 | L1 | L1/L2 | #AD/B | #AD/B | #AD/B |
| 1962 | AKC 122 | 16 | N2#121 | 5373:B8 | Dead | L1 | #AD/B | #AD/B | #AD/B | OK |
| 1388 | AKC 104 | 17 | N2#80 | 5022:C4 | L1/L2 | L1/L2 | L1/L2 | #AD/B | L1/L2 | L1/L2 |
| 1372 | AKC 123 | 18 | N2#79 | 5016:B8 | L1 | L1/L2 | #AD/B | < | #AD/B | #AD/˜B |
| 1402 | AKC 124 | 19 | N2#81 | 5033:D8 | #AD/B! | #AD/B! | L2/L3 | L4/#AD/B | L4/#AD/B | L4/#AD/B |
| 1396 | AKC 125 | 20 | N2#80 | 5031:G8 | L2/Dead(L3) | L2/Dead(L4) | L2 | L2 | #AD/B | < |

### EXAMPLE 3

### Nematicidal Activity of Anthelmintic Compositions 32-46

The C. elegans nematode activity assay for anthelmintic compounds 32-46 was similar to that described in Example 2 above, except for the following noted differences. The compound concentrations were adjusted to 140 μM and subjected to 2-fold dilutions to yield 140 μM, 70 μM, 35 μM, 17.5 μM, 8.8 μM, 4.4 μM, 2.2 μM, and 1.09 μM. The visual evaluation of viability was conducted at Day 4, and the results are presented in Table 2.

TABLE 2

| | μM Concentration | | | | | | | |
|---|---|---|---|---|---|---|---|---|
| Compound | 140 | 70 | 35 | 17.5 | 8.8 | 4.4 | 2.2 | 1.09 |
| AKC-138 | L1 | L1 | L1 | L2 | ˜B | OK | OK | OK |
| AKC-144 | L3/L4 | L4/AD/B | B | ˜B | OK | OK | OK | OK |
| AKC-141 | L1 | L1 | L1 | < | OK | OK | OK | OK |
| AKC-116 | L1/L2 | L2/L3 | L3 | B! | B | OK | OK | OK |
| AKC-117 | L1/L2 | L2/L3 | L3 | B! | B | < | OK | OK |
| AKC-118 | L2 | L2/L3 | L3 | L4/AD/B! | B | ˜B | OK | OK |
| Control | OK | OK | OK | OK | OK | OK | OK | OK |

### EXAMPLE 4

### Activity Against Nematode (C. elegans) Eggs

Compositions of the subject invention are surprisingly found to be ovicidal. The following procedures are used to test for lethal effects against nematode eggs.

Materials

As referred to herein, “S Medium” refers to “S basal” supplemented with CaCl₂, MgSO₄, and a trace metals solution as follow:

S basal

| | | |
|---|---|---|
| NaCl | 5.857 | g |
| 1 M potassium phosphate (pH 6) | 50.0 | ml |
| Cholesterol (5 mg/ml in EtOH) | 1.0 | ml |
| dH₂O | | |
| | 1 L | |

The above preparation is then autoclaved. S basal can be stored until needed.

Just prior to use, S Medium is made from S basal by adding, asceptically, the following components to 1L S basal (components should first be autoclaved separately):

| | |
|---|---|
| 1 M potassium citrate (pH 6) | 10 ml |
| Trace metals solution (see below) | 10 ml |
| 1M CaCl₂ | 3 ml |
| 1M MgSO₄ | 3 ml |

Trace Metals solution

| | | |
|---|---|---|
| Na₂EDTA | 1.86 g | (to 5 mM) |
| Fe₂SO₄.7H₂O | 0.69 g | (to 2.5 mM) |
| MnCl₂.4H₂O | 0.20 g | (to 1 mM) |
| ZnSO₄.7H₂O | 0.29 g | (to 1 mM) |
| CuSO₄.5H₂O | 0.025 g | (to 0.1 mM) |
| dH₂O | | |

Procedure:

1. Make anthelmintic compound dilutions as indicated in Examples 2-3.

2. To 500 μl of each dilution, added 10 μl of eggs (estimated>200 eggs/10 μl).

3. Mixed well and allowed to incubate at room temperature for from 30 minutes to 3 hours.

4. Centrifuge at 2000 rpm for 5 minutes at room temperature.

5. Pipette off supernatant.

6. Re-suspend in 500 μl S Medium.

7. Centrifuge at 2000 rpm for 5 minutes at room temperature

8. Pipette off supernatant.

9. Re-suspend in 300 μl S Medium.

10. Transfer 300 μl into 24-well tissue culture bioassay tray.

11. Add 2 μl of stationary phase E. coli to each well.

12. Score after 3 days at room temperature in the dark.

### EXAMPLE 5

### Additional Observations of Activity Against Nematode (C. elegans) Eggs

Additional tests are conducted to confirm the ovicidal activity. The following procedures are used.

1. Make anthelmintic compound dilutions to 2× concentrations shown in Example 4.

2. Distribute 0.5 ml of each dilution into 1.5-ml Eppendorf tubes.

3. Add 0.5 ml of C. elegans egg preparation to 0.5 ml 2× dilution to yield final exposure concentration.

4. Mix well and allow to incubate at room temperature for from 30 minutes to 3 hours.

5. Centrifuge at 2000 rpm for 5 minutes at room temperature.

6. Pipette off supernatant and re-suspend in 1.5 ml S Medium.

7. Spin as above for 2 minutes.

8. Pipette off supernatant and re-suspend in 1.5 ml S Medium.

9. Repeat #7.

10. Pipette off supernatant and re-suspend in 1.0 ml S Medium.

11. Add 280 μl of S Medium to each well of 24-well tissue culture plate.

12. Add 20 μl of each treated (and control) sample in triplicate into the respective wells.

13. Score after 3 days at room temperature in the dark.

### EXAMPLE 6

### Preparation of Anthelmintic Compounds 47 as specifically exemplified by compounds 32, and 48-118

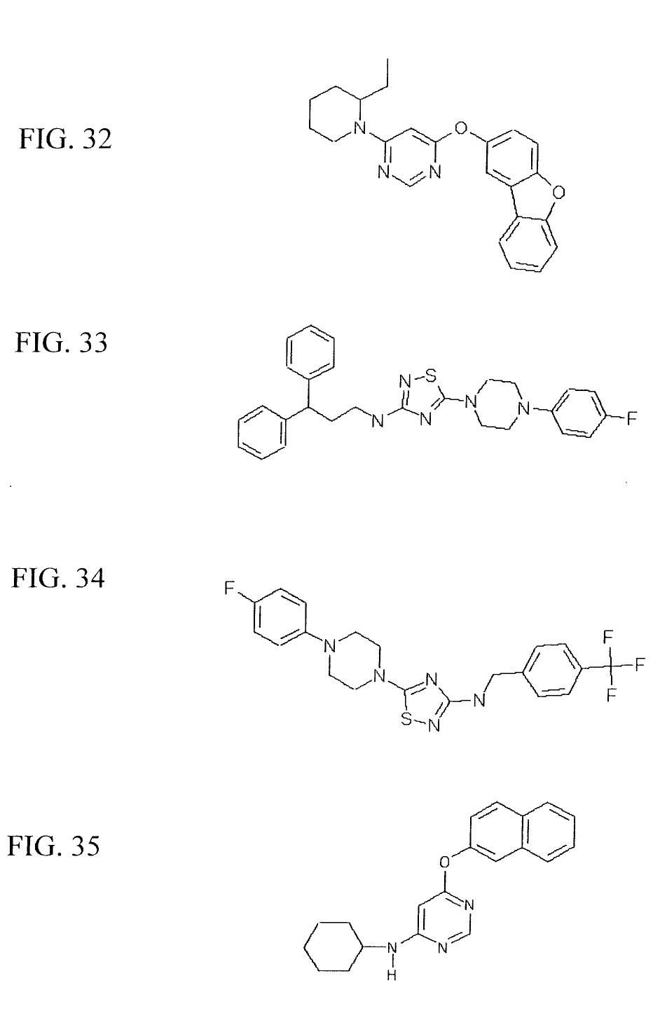

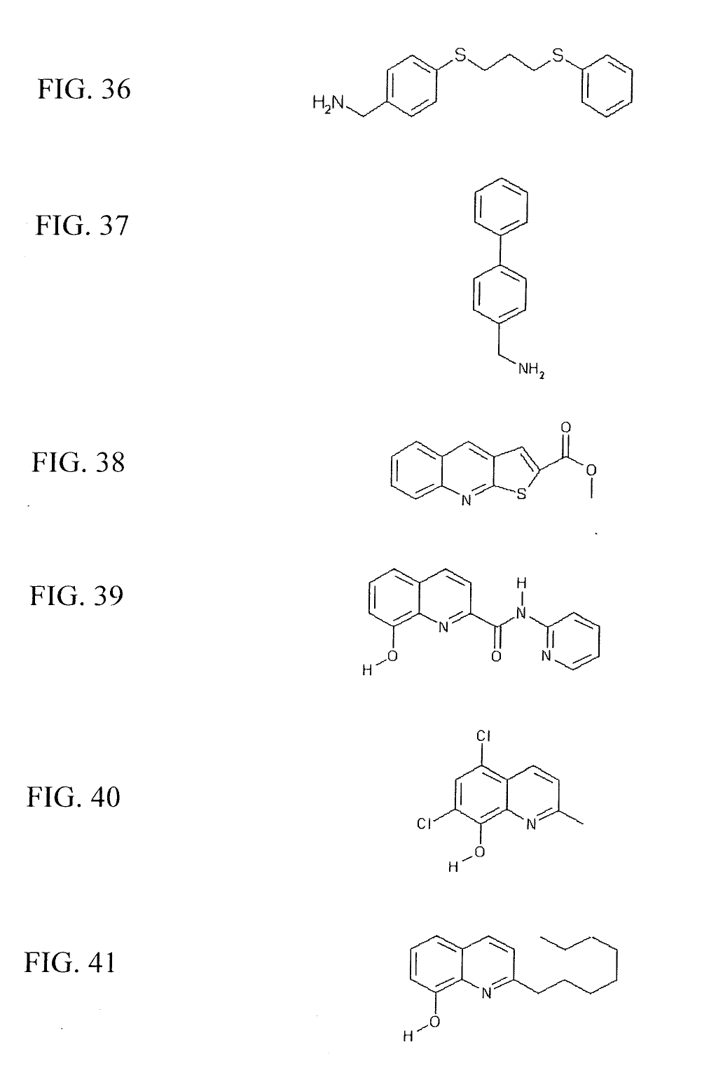

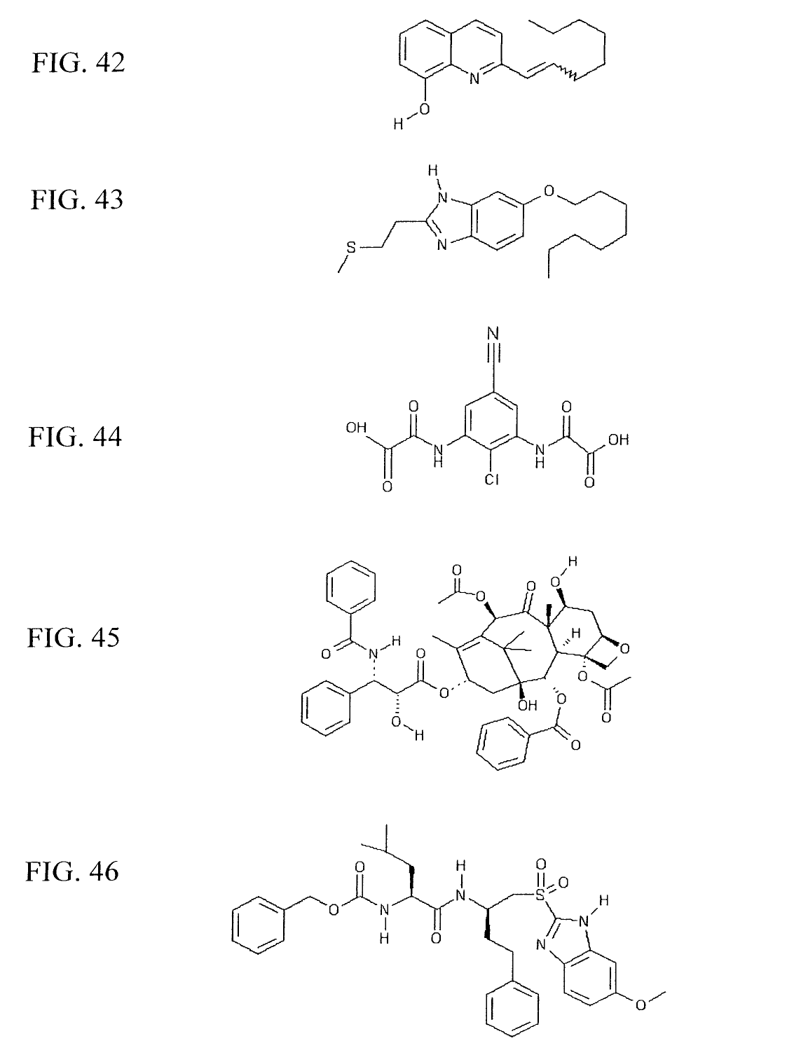

While the anthelmintic compounds of the subject invention can readily be produced using procedures well known to those skilled in the art, the following is a preferred method of producing anthelmintic Compounds 47, and exemplified Compounds 32 and 48-118, as shown in FIGS. 47, 32, and 48-118. The general library scheme resulting in Compounds 47 is depicted in FIG. 119.

The addition of the phenols to 4,6-dichloropyrimidine was done on a 5 mmol scale in standard glassware under nitrogen atmosphere. Sixteen 4-phenoxy-6-chloropyrimidines were made. On a small scale (0.1 mmol) hydrolysis of the dichloropyrimidine was a problem. Moisture was absorbed during the potassium t-butoxide and t-butanol transfers. Displacement of the second chloro was performed on the Gilson 215 liquid handler in septa capped 2-mi vials. Each of 16 mono-chloropyrimidines was distributed into 48 vials and treated with each of 48 amines. Each reaction mixture was extracted with buffered water in Chem Elut tubes.

For 4-(2′-naphthoxy)-6-chloropyrimidine, 2-Naphthol (734 mg, 5.1 mmols) was dissolved in 6-ml of warm 2-methyl-2-propanol and under nitrogen was treated all at once with 7.5-ml of a 1.0 M solution of potassium tert-butoxide in 2-methyl-2-propanol. Reaction mixture became opaque. A solution of 4,6-dichloropyrimidine (775 mg, 5.2 mmols) in warm 2-methyl-2-propanol (8.6-ml) was added and the mixture was heated to 35° C. RPHPLC or TLC (1/1 hexane/ethyl acetate) was used to monitor the reaction. After 3 hours, if excess phenol was present then additional 4,6-dichloropyrimidine was added. The mixture was stirred over night. The mixture was then diluted with ether and washed with water (2×). The aqueous layers were combined and extracted with ether. The combined ether layers were washed with brine, dried over magnesium sulfate and concentrated under vacuum. 16 phenols were used. 4-pyridone failed to give a good yield of clean material under these conditions.

The library was made in two sets of 384 compounds on the Gilson 215 Liquid Handler. Fifteen 4-chloro-6-phenoxy-pyrimidines (5 mmoles made up to 10-ml) were dissolved in dichloroethane (DCE). The 8-hydroxy quinoline (ACD 6807) derivative was dissolved in 6-ml DCE and made up to 10-ml with acetonitrile and kept at 35° C. Each chloropyrimidine solution (200 μl, 0.1 mmol) was transferred to each of 24 septa capped 2-ml vials held in four 16×6 Gilson racks. The vial caps were loosened and the solvent evaporated under vacuum at 40° C. for 20 hours. Each of 24 amines (4 mmoles) was combined with 4 mmols (556 μl) of triethyl amine and diluted to 5.5-ml with a 9/1 mixture of 2-methyl-2-propanol/DCE. Each amine (300 μl, 0.218 mmol) was transferred to 16 vials containing the different monochloropyrimidines. The racks were heated to 85° C. for 72 hours. The vials treated with sec-butyl amine (ACD 8094) and cyclohexyl amine (ACD 1486) were retreated with amine and heated for 24 hours. The test library results indicated that these volatile amines evaporated before complete reaction.

The reaction vial caps were loosened and the solvent was evaporated under vacuum at 40° C. Chem Elut columns (3 cc) were treated with 300μl of 1N hydrochloric acid. The “Chem Elut” columns for ACD6367, ACD9819, ACD6412 and ACD 6216 were treated with water in place of acid. The reaction mixture was dissolved in 400 μl chloroform and transferred by the Gilson 215 Liquid Handler to the “Chem Elut” columns. The columns were eluted with 2-ml of methylene chloride. The solvent was evaporated under vacuum. The library was dissolved in DMSO for plating.

### EXAMPLE 7

### Nematicidal Activity of Anthelmintic Compositions 48-118

The nematicidal activity of anthelmintic Compositions 48-118 were determined in accordance with the procedure outlined in Example 2. The results are reported in Table 3.

TABLE 3

| HTS Data | | | |
|---|---|---|---|
| HTS Tracking | Initial HTS Run | Follow-up HTS Run | 5 Day Visual Score |

| AKC # | MP # | Well | mOD | % Run | Visual Score | mOD | % Run | Visual Score | Well Add | V1 | V2 | V3 | V4 | V5 | V6 |
|---|---|---|---|---|---|---|---|---|---|---|---|---|---|---|---|
| 339 | 2665 | F2 | 277 | 142% | 1AD/B | 153 | 113% | B | 2665:F2 | OK | OK | OK | OK | OK | OK |
| 109 | 2665 | B5 | 258 | 132% | 1AD/B! | 163 | 121% | L4/AD/B! | 2665:B5 | L1 | L1 | #AD/B! | #AD/B! | #AD/B | B |
| 148 | 2665 | F6 | 278 | 143% | Dead-Black Husk | 170 | 126% | Dead | 2665:F6 | Dead | Dead | Dead | #AD/B | ˜B | ˜B |
| 341 | 2666 | D2 | 195 | 100% | 4AD/˜B | 137 | 101% | B! | 2666:D2 | #AD/B! | #AD/B | #AD/B | ˜B | ˜B | OK |
| | | | | | | | | | | Dead | L1 | L1 | L1/L2 | #AD/B | OK |
| 342 | 2666 | A4 | 207 | 106% | L4/AD/B! | 124 | 92% | B!-Healthy | 2666:A4 | L1/L2 | L1/L2 | #AD/B | ˜B | OK | OK |
| 343 | 2666 | B4 | 251 | 129% | L3/L4-Clear | 166 | 123% | L4/AD/B!-No P | 2666:B4 | #AD/B! | #AD/B! | #AD/B! | B | OK | OK |
| 344 | 2666 | D4 | 256 | 131% | L2 | 161 | 119% | L3/L4 | 2666:D4 | Dead | Dead | #AD/B | OK | OK | OK |
| 345 | 2666 | D5 | 264 | 135% | L1 | 154 | 114% | L1/L2 | 2666:D5 | L1 | L1 | L4 | OK | OK | OK |
| 346 | 2666 | E5 | 258 | 132% | 2AD/B | 155 | 115% | L4/1AD/B | 2666:E5 | L1 | L1 | #AD/B | #AD/B | OK | OK |
| 347 | 2666 | A10 | 216 | 111% | Dead | 143 | 106% | L1/1L3 | 2666:A10 | Dead | L1 | L1 | #AD/B | OK | OK |
| 348 | 2666 | B10 | 246 | 126% | Dead | 166 | 123% | L1/L2 | 2666:B10 | Dead | Dead | Dead | #AD/B | < | B |
| | | | | | | | | | | #AD/B! | #AD/B! | B! | OK | OK | OK |
| 349 | 2667 | B3 | 247 | 127% | 2AD/B! | 139 | 103% | B | 2667:B3 | #AD/B | OK | #AD/˜B | OK | OK | OK |
| 350 | 2667 | A5 | 245 | 126% | Dead | 136 | 101% | Dead | 2667:A5 | L1 | L1 | #AD/B | OK | OK | OK |
| 351 | 2667 | B5 | 263 | 135% | 1AD/B | 156 | 116% | Dead | 2667:B5 | L1 | #AD/B | < | < | OK | OK |
| 352 | 2667 | H5 | 228 | 117% | 2AD/B | 128 | 95% | ˜B | 2667:H5 | Dead | OK | OK | OK | OK | OK |
| 353 | 2667 | F6 | 276 | 142% | Dead | 173 | 128% | L1 | 2667:F6 | Dead | Dead | Dead | L1/L2 | OK | OK |
| 354 | 2667 | G7 | 267 | 137% | 1AD/B | 146 | 108% | B | 2667:G7 | Dead | #AD/B | #AD/˜B | < | OK | < |
| 149 | 2668 | F5 | 259 | 133% | Dead | 135 | 100% | Dead | 2668:F5 | Dead | Dead | Dead | #AD/B | < | < |
| | | | | | | | | | | L1 | L1 | L1 | #AD/B! | < | OK |
| 355 | 2668 | D6 | 251 | 129% | Dead | 134 | 99% | L3/L4 | 2668.D6 | Dead | #AD/B | #AD/B! | ˜B | OK | < |
| 356 | 2668 | G6 | 272 | 139% | Dead | 165 | 122% | L2 | 2668:G6 | Dead | Dead | #AD/B | OK | OK | < |
| 357 | 2668 | B7 | 224 | 115% | 3AD/B | 132 | 98% | L4/AD/B! | 2668:B7 | Dead | #AD/B | #AD/B | OK | OK | OK |
| 358 | 2668 | D7 | 252 | 129% | Dead | 153 | 113% | L3-Dead L3 | 2668:D7 | Dead | Dead | #AD/B! | OK | OK | OK |
| 359 | 2668 | E7 | 271 | 139% | Dead | 135 | 100% | L3-Dead L3 | 2668:E7 | Dead | Dead | #AD/B! | B | OK | OK |
| 360 | 2668 | H7 | 272 | 139% | 1AD/B!-No P | 151 | 112% | L4/AD/B! | 2668:H7 | Dead | Dead | OK | OK | OK | OK |
| 361 | 2669 | D4 | 242 | 124% | 2AD/B | 133 | 99% | B | 2669:D4 | Dead | #AD/B | #AD/˜B | < | < | < |
| | | | | | | | | | | B! | B! | B! | OK | OK | OK |
| 362 | 2669 | D10 | 183 | 94% | 5AD/˜B | 121 | 90% | B | 2669:D10 | Dead | #AD/B | < | OK | OK | OK |
| 363 | 2670 | F3 | 275 | 141% | Dead | 165 | 122% | Dead | 2670:F3 | Dead | < | ˜B | OK | OK | OK |
| 364 | 2670 | G6 | 236 | 121% | 4AD/˜B | 146 | 108% | L4/AD/B! | 2670:G6 | Dead(L2/L3) | #AD/B | ˜B | < | OK | OK |
| 365 | 2670 | D7 | 256 | 131% | Dead | 139 | 103% | L3/L4/AD/B! | 2670:D7 | Dead | #AD/B | B | OK | OK | OK |
| 366 | 2670 | G10 | 260 | 133% | 1AD/B | 156 | 116% | 2AD/B! | 2670:G10 | #AD/B | #AD/B | OK | OK | OK | OK |
| 367 | 2670 | D11 | 239 | 123% | 2AD/B | 123 | 91% | B | 2670:D11 | L1 | < | < | < | OK | OK |
| 368 | 2671 | G1 | 238 | 122% | 1AD/B | 105 | 78% | B | 2671:G1 | Dead | #AD/B | #AD/B | < | OK | OK |
| | | | | | | | | | | L1 | L1 | L1 | L1 | OK | < |
| 369 | 2671 | D2 | 225 | 115% | 1AD/B | 127 | 94% | B | 2671:D2 | Dead | #AD/B | < | OK | OK | OK |
| 370 | 2671 | E2 | 220 | 113% | 4AD/B | 131 | 97% | B | 2671:E2 | Dead | #AD/B | OK | OK | OK | OK |
| 371 | 2671 | A5 | 237 | 122% | Dead | 143 | 106% | L1/L2 | 2671:A5 | Dead | #AD/B | OK | OK | OK | OK |
| 372 | 2671 | G5 | 279 | 143% | Dead | 154 | 114% | L1/L2 | 2671:G5 | L1 | #AD/B | OK | OK | OK | OK |
| 373 | 2671 | D6 | 257 | 132% | L1/L2 | 112 | 83% | B | 2671:D6 | L4 | #AD/B | #AD/B | OK | OK | OK |
| 374 | 2671 | H6 | 271 | 139% | L3-Dying | 154 | 114% | L2/L3 | 2671:H6 | Dead(L2/L3) | #AD/B | OK | OK | OK | OK |
| 375 | 2671 | D7 | 231 | 118% | 2AD/B | 125 | 93% | B | 2671:D7 | Dead | L1/L2 | #AD/B | OK | OK | < |
| | | | | | | | | | | B! | #AD/B! | B! | OK | OK | OK |
| 376 | 2671 | G7 | 256 | 131% | 2AD/B | 155 | 115% | L2/L3/1AD | 2671:G7 | L1 | OK | OK | OK | OK | OK |
| 377 | 2672 | A4 | 220 | 113% | 3AD/B | 147 | 109% | L4/AD/B!-No P | 2672:A4 | L1 | #AD/B | OK | OK | OK | OK |
| 378 | 2672 | D6 | 250 | 128% | Dead | 135 | 100% | L3/L4/1AD/B! | 2672:D6 | Dead | #AD/B | < | OK | OK | OK |
| 379 | 2672 | G6 | 277 | 142% | Dead/1AD | 144 | 107% | L4/AD/B! | 2672:G6 | Dead | #AD/B | OK | OK | OK | OK |
| 380 | 2672 | D7 | 251 | 129% | Dead | 138 | 102% | L4/AD/B! | 2672:D7 | L1 | #AD/B | OK | OK | OK | OK |
| 381 | 2672 | E7 | 270 | 138% | Dead/1AD/B! | 147 | 109% | L4/AD/B! | 2672:E7 | L1 | ˜B | < | OK | OK | OK |
| 382 | 2672 | F7 | 275 | 141% | Dead | 159 | 118% | L3/L4 | 2672:F7 | Dead | #AD/B | OK | OK | OK | OK |
| | | | | | | | | | | L1 | L1 | L1/L2 | L2/L3 | OK | OK |
| 383 | 2672 | H7 | 257 | 132% | 5AD/B | 149 | 110% | L4/AD/B! | 2672:H7 | L1 | ˜B | OK | OK | OK | OK |
| 384 | 2672 | D8 | 264 | 135% | Dead | 141 | 104% | L4/AD/B! | 2672:D8 | Dead | #AD/B | OK | OK | OK | OK |
| 385 | 2672 | F8 | 270 | 138% | L2/L3 | 157 | 116% | L3/L4 | 2672:F8 | L1/L2 | #AD/B | OK | OK | OK | OK |
| 386 | 2672 | G8 | 281 | 144% | Dead | 147 | 109% | L3 | 2672:G8 | L1 | L1 | #AD/B | ˜B | OK | OK |
| 387 | 2672 | D9 | 250 | 128% | Dead | 124 | 92% | L2/L3 | 2672:D9 | Dead | #AD/B | #AD/˜B | ˜B | OK | OK |
| 388 | 2672 | E9 | 239 | 123% | 4AD/B | 133 | 99% | B | 2672:E9 | Dead | #AD/B | OK | OK | OK | OK |
| 389 | 2672 | F9 | 272 | 139% | Dead | 147 | 109% | 2AD/B! | 2672:F9 | Dead | L1 | OK | OK | OK | OK |
| | | | | | | | | | | #AD/B! | B! | B! | OK | OK | OK |
| 390 | 2672 | H9 | 260 | 133% | B! | 148 | 110% | L4 | 2672:H9 | L1 | #AD/B! | OK | OK | OK | OK |
| 391 | 2673 | A1 | 230 | 118% | Dead | 144 | 107% | Dead | 2673:A1 | Dead | OK | OK | OK | OK | OK |
| 392 | 2673 | B1 | 233 | 119% | Dead | 143 | 106% | Dead | 2673:B1 | Dead | #AD/B | OK | OK | OK | OK |
| 393 | 2673 | C1 | 288 | 148% | Dead | 169 | 125% | Dead | 2673:C1 | Dead | #AD/˜B | OK | OK | OK | OK |
| 394 | 2673 | D1 | 264 | 135% | Dead | 150 | 111% | Dead | 2673:D1 | Dead | Dead | #AD/B | OK | OK | < |
| 395 | 2673 | E1 | 247 | 127% | Dead | 157 | 116% | Dead | 2673:E1 | Dead | Dead | #AD/B | OK | OK | < |
| 150 | 2673 | F1 | 265 | 136% | Dead | 151 | 112% | Dead | 2673:F1 | Dead | Dead | Dead | Dead | OK | OK |
| | | | | | | | | | | L1 | L1 | L1 | L1/L2 | OK | OK |
| 396 | 2673 | G1 | 266 | 136% | Dead | 152 | 113% | Dead | 2673:G1 | Dead | Dead | < | OK | OK | OK |
| 397 | 2673 | H1 | 282 | 145% | Dead | 158 | 117% | Dead | 2673:H1 | OK | OK | OK | OK | OK | OK |
| 398 | 2673 | A2 | 231 | 118% | Dead | 126 | 93% | Dead | 2673:A2 | Dead | Dead | #AD/˜B | OK | < | OK |
| 399 | 2673 | B2 | 248 | 127% | Dead | 181 | 134% | Dead | 2673:B2 | Dead | Dead | OK | OK | OK | OK |
| 400 | 2673 | C2 | 256 | 131% | Dead | 126 | 93% | Dead | 2673:C2 | Dead | #AD/˜B | OK | OK | OK | OK |
| 401 | 2673 | D2 | 281 | 144% | Dead | 165 | 122% | Dead | 2673:D2 | Dead | Dead | #AD/B! | OK | OK | OK |
| 151 | 2673 | E2 | 329 | 169% | Dead | 161 | 119% | Dead | 2673:E2 | Dead | Dead | Dead | #AD/B | < | < |
| | | | | | | | | | | B! | B! | B! | OK | OK | OK |
| 402 | 2673 | F2 | 270 | 138% | Dead | 160 | 119% | Dead | 2673:F2 | Dead | Dead | OK | OK | OK | OK |
| 403 | 2673 | G2 | 280 | 144% | Dead | 175 | 130% | Dead | 2673:G2 | #AD/B | #AD/˜B | OK | OK | OK | OK |
| 404 | 2673 | H2 | 302 | 155% | Dead | 168 | 124% | Dead | 2673:H2 | Dead | Dead | OK | OK | OK | OK |
| 405 | 2673 | F6 | 274 | 141% | 4AD/B | 162 | 120% | 1AD/B! | 2673:F6 | L1 | L1 | #AD/˜B | OK | OK | OK |

### EXAMPLE 8

### Sheep Test I Experimental Procedure

Sheep naturally infected with a variety of gastrointestinal nematodes are purchased from local sources and are transported to the test site. The animals are housed in a manner to preclude further infection by nematode larvae. The animals are evaluated for the presence of adequate nematode burdens by performing a standard fecal egg per gram (EPG) count. Eggs are differentiated into the following groups: trichostrongyle (strongyle), Strongyloides, Trichuris, or Nematodinis. Only sheep judged by the study parasitologist to have adequate nematode infections are retained as test subjects.

The sheep are fed good quality hay (no concentrated rations) and water ad libitum. Following a five-day acclimation period, the sheep are randomly assigned by EPG count into treatment groups which include non-treated Negative control (placebo); Positive Control (commercially available ivermectin for sheep): and various anthelmintic compounds of the present invention (test compound) dissolved in DMSO. The first replicate of 10 animals is randomly assigned to groups 1-10; the second replicate of 10 animals is randomly assigned to groups 1-10; and the third replicate of 10 animals is randomly assigned to groups 1-10. Thus 10 groups of 3 animals each is created.

The randomization is performed on fecal samples collected 24-48 hours prior to scheduled treatment. The EPG counts are performed according to Zimmerman Research SOP # NMEPG.99.01

On treatment day, the animals are weighed and divided into groups with three animals per group as follows:

GROUP 1: Non-treated negative control (placebo) of 10 ml of DMSO.

GROUP 2: Positive Control treatment of 200 mcg/kg commercially available ivermectin for sheep.

GROUP 3: Compound @ dissolved in DMSO.

GROUP 4: Compound @ dissolved in DMSO.

GROUP 5: Compound @ dissolved in DMSO.

GROUP 6: Compound @ dissolved in DMSO.

GROUP 7: Compound @ dissolved in DMSO.

GROUP 8: Compound @ dissolved in DMSO.

GROUP 9: Compound @ dissolved in DMSO.

GROUP 10: Compound @ dissolved in DMSO.

The placebo (DMSO), the commercially available drug, and the test anthelmintic compounds are administered in a 3 ml volume by subcutaneous injection using a sterile syringe fitted with a proper needle. The animal is adequately immobilized for injection of the placebo, commercially available drug, or test anthelmintic compound.

Following treatment, the animals are observed at hourly intervals for the first 8 hours, then daily until necropsy. They will continue to be housed in a manner to prevent further nematode infections. Fecal samples are taken for EPG counts on the 5th day and 7th day after treatment.

Seven days following treatment the sheep are humanely slaughtered in accordance with the Guide for the Care and Use of Laboratory Animals (DHEW Publication No. 86-23). Necropsy procedures are according to Zimmerman Research SOP # NCRGIH.99.01, Necropsy for Helminth Recovery, specifically for gastrointestinal nematodes. Fecal samples are taken for EPG counts during the sample collection process on this day. All animals are necropsied, but only the animals from the experimental treatment groups that have a significant egg count reduction on day 5 or day 7 will have intestinal material collected for nematode recovery and identification.

Nematodes are recovered, identified, and enumerated according to Zimmerman Research SOP # NEMRECOVID.99.01. All individuals performing nematode recoveries are blinded to treatment versus control animals. Preliminary estimates of total nematodes recovered from each gut sample are provided prior to identification and enumerations by the study parasitologist. At the discretion of the study parasitologist, seven days after the drug administration fecal egg counts are performed and all animals showing 90% or better trichostrongylid egg reduction will be slaughtered using humane methods recommended by the AVMA. The neck blood vessels are severed and after the animal is completely exsanguinated, the abdomen are opened. The abomasum, the small and large intestines are tied at the omasal and pyloric openings, the duodenum, the end of the small intestine and at the end of the large intestine. Each section is transferred in a separate bucket containing warm water and is slit open and thoroughly washed. The epithelium is inspected before it is removed. The thus prepared washings are saved in gallon jars. An appropriate preservative is added. If preservative is not available, all the intestinal washing should kept in a refrigerator. These washings are passed through a 100-mesh sieve (pore size 149 pm), and the residue is examined for the presence of worms under a dissecting microscope, Lugol's solution may be used to stain the worms. All worms are picked up counted and identified as to the species. An effort should be made to recover any immature forms present. The efficacy should be calculated using the controlled anthelmintic test.

$$Percentage efficacy = \frac{\begin{matrix}( Mean number of worms in controls minus \\ Mean number of worms in treated animal )\end{matrix}}{Mean number of worms in controls} \times 100$$

Results are depicted in Tables 4 and 5.

TABLE 4

| | | Sheep | Trial | 17-Jan | | |
|---|---|---|---|---|---|---|
| | AKK 101 | Rumen | Injection | Worm | Counts | |
| | Sheep | Weight/lbs | Abomasum | Abomasum | Small Intestine | Small Intest. |
| Akkadix | Number | 1/17/2000 | Haemonchus | ostertagia | Trichostrongylus | Nematodirus |
| Group | 81 | 84 | 420 | 60 | 0 | 146 |
| Negative | 751 | 116 | 500 | 860 | 320 | 2300 |
| Control | 56 | 871 | 0 | 0 | 80 | 0 |
| Mean Ct. | | | 307 | 307 | 133 | 813 |
| Group | 83 | 98 | 0 | 0 | 0 | 0 |
| Ivermectin | 92 | 129 | 0 | 0 | 0 | 0 |
| 200 mcg/kg | 53 | 82 | 0 | 0 | 0 | 0 |
| Mean Ct | | | 0 | 0 | 0 | 0 |
| | | % Efficacy | 100 | 100 | 100 | 100 |
| Group | 77 | 80 | 20 | 20 | 540 | 100 |
| AKC 109 | 93 | 100 | 1500 | 0 | 160 | 500 |
| 1.4 mg/kg | 70 | 65 | 0 | 0 | 0 | 460 |
| Mean Ct | | | 507 | 7 | 233 | 353 |
| | | % Efficacy | | 98 | | 57 |

TABLE 5

| | Trial -1 | Sheep | AKK 101 | Strongyles | Strongyles | | Strongyles | |
|---|---|---|---|---|---|---|---|---|
| | Sheep | Weight/lbs | Total | 17-Jan | 22-Jan | | 24-Jan | |
| Akkadix | Number | 1/12/2000 | EPG-pre | EPG-pre | EPG-5 day | % Change | EPG-7 day | % Change |
| Group | 77 | 72 | 2420 | 2420 | 30 | | 380 | 84.30 |
| AKC 109 | 93 | 90 | 440 | 200 | 590 | | NS | −195.00 |
| 1.4 mg/kg | 70 | 63 | 110 | 10 | 0 | | 0 | 100.00 |
| Total/Mean | | 225 | 990.00 | 876.67 | 206.67 | 76.43 | 126.67 | 85.55 |
| Group | 83 | 91 | 2310 | 2000 | 10 | | 0 | 100.00 |
| Ivermectin | 92 | 113 | 570 | 570 | 0 | | 0 | 100.00 |
| .2 mg/kg | 53 | 77 | 90 | 70 | 0 | | 0 | 100.00 |
| Total/Mean | | 281 | 990.00 | 880.00 | 3.33 | 99.62 | 0.00 | 100.00 |
| Group | 81 | 74 | 2240 | 2240 | 780 | | 770 | 65.63 |
| Negative | 75 | 109 | 370 | 300 | 260 | | 1360 | −353.33 |
| Control | 56 | 80 | 40 | 40 | 30 | | 30 | 25.00 |
| Total/Mean | | 263 | 883.33 | 860.00 | 356.67 | 58.53 | 720.00 | 16.28 |

### EXAMPLE 9

### Sheep Test II Experimental Procedure

Sheep naturally infected with a variety of gastrointestinal nematodes are purchased from local sources and are transported to the test site. The animals are housed in a manner to preclude further infection by nematode larvae. The animals are evaluated for the presence of adequate nematode burdens by performing a standard fecal egg per gram (EPG) count. Eggs are differentiated into the following groups: trichostrongyle (strongyle), Strongyloides, Trichuris, or Nematodiris. Only sheep judged by the study parasitologist to have adequate nematode infections are retained as test subjects.

The sheep are fed good quality hay (no concentrated rations) and water ad libitum. Following a five day acclimation period, the sheep are randomly assigned by EPG count into the following treatment groups: Groups 1-9, various anthelmintic compounds of the present invention (test compound) dissolved in DMSO: Group 10, Positive Control (commercially available ivermectin for sheep); Group 11, non-treated Negative control (DMSO only). The first replicate of 1 I animals is randomly assigned to groups 1-1; the second replicate of 11 animals is randomly assigned to groups 1-11; and the third replicate of 11 animals is randomly assigned to groups 1-1. Thus 11 groups of 3 animals each are created.

The randomization is performed on fecal samples collected 24-48 hours prior to scheduled treatment. The EPG counts are performed according to Zimmerman Research SOP # NMEPG.99.01.

GROUP 1: AKKADIX compound dissolved in DMSO.

GROUP 2: AKKADIX compound dissolved in DMSO.

GROUP 3: AKKADIX compound dissolved in DMSO.

GROUP 4: AKKADIX compound dissolved in DMSO.

GROUP 5: AKKADIX compound dissolved in DMSO.

GROUP 6: AKKADIX compound dissolved in DMSO.

GROUP 7: AKKADIX compound dissolved in DMSO.

GROUP 8: AKKADIX compound dissolved in DMSO.

GROUP 9: AKKADIX compound dissolved in DMSO.

GROUP 10: Positive Control treatment of 200 mcg/kg commercially available ivermectin for sheep.

GROUP 11: Non-treated negative control (placebo) of 3 ml of DMSO.

On treatment day, the animals are weighed, tagged, and divided into groups of three animals per group as follows:

The placebo (DMSO), the commercially available drug, and the test anthelmintic compounds are administered in a 3 ml volume of DMSO by subcutaneous injection using a sterile syringe fitted with a sterile needle. The site of injection is clipped and swabbed with alcohol prior to injection. The animal is adequately immobilized for injection of the placebo, commercially available drug, or experimental compound.

Following treatment, the animals are observed at hourly intervals for the first 8 hours, then daily until necropsy. They are housed in a manner to prevent further nematode infections.

On the fifth day following treatment, fecal samples are obtained from each animal, properly labeled and used for EPG counts.

Seven days following treatment, all the sheep are weighed and humanely slaughtered in accordance with the Guide for the Care and Use of Laboratory Animals (DHEW Publication No. 86-23). Necropsy procedures are according to Zimmerman Research SOP # NCRGIH.00.01, Necropsy for Helminth Recovery, specifically for gastrointestinal nematodes. Fecal samples are taken for EPG counts during the sample collection process on this day.

Nematodes are recovered, identified, and enumerated according to Zimmerman Research SOP # NEMRECOVID.00.01. All individuals performing nematode recoveries are blinded to treatment versus control animals.

Anthelmintic efficacy is calculated using the controlled test procedure:

$$\% Efficacy = \frac{\begin{matrix}Mean number of worms in controls minus \\ mean number of worms in treated \end{matrix}}{Mean number of worms in controls} \times 100$$

Results are depicted in Tables 6 and 7.

TABLE 6
24-May

| | AKK-102 | Sheep | Worm Counts | | | | |
|---|---|---|---|---|---|---|---|
| | Sheep | Weight/lbs | Abomasum | Abomasum | Small Intestine | Small Intest. | Large Intest. |
| Akkadix | Number | 5/17/2000 | Haemonchus | Ostertagia | Trichostrongylus | Nematodirus | Trichuris |
| Group | 530 | 47 | 60 | 580 | 40 | 8500 | 5 |
| Negative | 1341 | 57 | 60 | 20 | 20 | 2520 | 15 |
| Control | 524 | 54 | 20 | 80 | 0 | 0 | 10 |
| Mean Ct. | | | 47 | 227 | 20 | 3673 | 10 |
| Group | 1347 | 45 | 0 | 0 | 0 | 100 | 0 |
| Ivermectin | 1336 | 58 | 20 | 0 | 0 | 100 | 0 |
| 200 mcg/kg | 539 | 47 | 0 | 0 | 0 | 0 | 0 |
| Mean Ct. | | | 7 | 0 | 0 | 67 | 0 |
| | | % Efficacy | 86 | 100 | 100 | 98 | |
| Group | 538 | 38 | 0 | 1100 | 240 | 12020 | 5 |
| AKC 109 | 1346 | 43 | 0 | 280 | 20 | 2220 | 0 |
| .8 mg/kg | Mean Ct. | | 0 | 690 | 130 | 7120 | 3 |
| | | % Efficacy | 100 | | | | |

TABLE 7

| | Trial -2 | Sheep | AKK 101 | Strongyles | Strongyles | | Strongyles | |
|---|---|---|---|---|---|---|---|---|
| | Sheep | Weight/lbs | Total | 15-May | 22-May | | 24-May | |
| Akkadix | Number | 5/17/2000 | EPG-pre | EPG-pre | EPG-5 day | % Change | EPG-7 day | % Change |
| Group | 538 | 38 | 1310 | 610 | 360 | | 220 | 63.93 |
| AKC 109 | 1346 | 43 | 420 | 400 | 130 | | 280 | 30.00 |
| .8 mg/kg | | | | | | | | |
| Total/Mean | | 81 | 865.00 | 505.00 | 245.00 | 51.49 | 250.00 | 50.50 |
| Group | 530 | 47 | 710 | 640 | 690 | | 300 | 53.13 |
| Negative | 1341 | 57 | 160 | 120 | 160 | | 340 | −183.33 |
| Control | 524 | 54 | 70 | 60 | 140 | | 50 | 16.67 |
| Total/Mean | | 158 | 313.33 | 273.33 | 330.00 | −20.73 | 230.00 | 15.85 |
| Group 1 | 1347 | 45 | 560 | 450 | 0 | | 0 | 100.00 |
| Ivermectin | 1336 | 58 | 220 | 170 | 0 | | 0 | 100.00 |
| 200 mcg/kg | 539 | 47 | 100 | 40 | 0 | | 0 | 100.00 |
| Total/Mean | | 150 | 293.33 | 220.00 | 0.00 | 100.00 | 0.00 | 100.00 |

It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A method for controlling nematodes which comprises contacting said nematodes with a nematode-controlling effective amount of a composition comprising at least one compound having Structure 47.

2. A method for controlling nematodes which comprises contacting said nematodes with a nematode-controlling effective amount of a composition comprising at least one compound having a structure selected from the group consisting of Structures 48-118.

3. The method of claim 2, wherein said compound is Compound 32.

4. The method of claim 2, wherein said compound is Compound 48.

5. The method of claim 2, wherein said compound is Compound 49.

6. The method of claim 2, wherein said compound is Compound 50.

7. The method of claim 2, wherein said compound is Compound 51.

8. The method of claim 2, wherein said compound is Compound 52.

9. The method of claim 2, wherein said compound is Compound 53.

10. The method of claim 2, wherein said compound is Compound 54.

11. The method of claim 2, wherein said compound is Compound 55.

12. The method of claim 2, wherein said compound is Compound 56.

13. The method of claim 2, wherein said compound is Compound 57.

14. The method of claim 2, wherein said compound is Compound 58.

15. The method of claim 2, wherein said compound is Compound 59.

16. The method of claim 2, wherein said compound is Compound 60.

17. The method of claim 2, wherein said compound is Compound 61.

18. The method of claim 2, wherein said compound is Compound 62.

19. The method of claim 2, wherein said compound is Compound 63.

20. The method of claim 2, wherein said compound is Compound 64.

21. The method of claim 2, wherein said compound is Compound 65.

22. The method of claim 2, wherein said compound is Compound 66.

23. The method of claim 2, wherein said compound is Compound 67.

24. The method of claim 2, wherein said compound is Compound 68.

25. The method of claim 2, wherein said compound is Compound 69.

26. The method of claim 2, wherein said compound is Compound 70.

27. The method of claim 2, wherein said compound is Compound 71.

28. The method of claim 2, wherein said compound is Compound 72.

29. The method of claim 2, wherein said compound is Compound 73.

30. The method of claim 2, wherein said compound is Compound 74.

31. The method of claim 2, wherein said compound is Compound 75.

32. The method of claim 2, wherein said compound is Compound 76.

33. The method of claim 2, wherein said compound is Compound 77.

34. The method of claim 2, wherein said compound is Compound 78.

35. The method of claim 2, wherein said compound is Compound 79.

36. The method of claim 2, wherein said compound is Compound 80.

37. The method of claim 2, wherein said compound is Compound 81.

38. The method of claim 2, wherein said compound is Compound 82.

39. The method of claim 2, wherein said compound is Compound 83.

40. The method of claim 2, wherein said compound is Compound 84.

41. The method of claim 2, wherein said compound is Compound 85.

42. The method of claim 2, wherein said compound is Compound 86.

43. The method of claim 2, wherein said compound is Compound 87.

44. The method of claim 2, wherein said compound is Compound 88.

45. The method of claim 2, wherein said compound is Compound 89.

46. The method of claim 2, wherein said compound is Compound 90.

47. The method of claim 2, wherein said compound is Compound 91.

48. The method of claim 2, wherein said compound is Compound 92.

49. The method of claim 2, wherein said compound is Compound 93.

50. The method of claim 2, wherein said compound is Compound 94.

51. The method of claim 2, wherein said compound is Compound 95.

52. The method of claim 2, wherein said compound is Compound 96.

53. The method of claim 2, wherein said compound is Compound 97.

54. The method of claim 2, wherein said compound is Compound 98.

55. The method of claim 2, wherein said compound is Compound 99.

56. The method of claim 2, wherein said compound is Compound 100.

57. The method of claim 2, wherein said compound is Compound 101.

58. The method of claim 2, wherein said compound is Compound 102.

59. The method of claim 2, wherein said compound is Compound 103.

60. The method of claim 2, wherein said compound is Compound 104.

61. The method of claim 2, wherein said compound is Compound 105.

62. The method of claim 2, wherein said compound is Compound 106.

63. The method of claim 2, wherein said compound is Compound 107.

64. The method of claim 2, wherein said compound is Compound 108.

65. The method of claim 2, wherein said compound is Compound 109.

66. The method of claim 2, wherein said compound is Compound 110.

67. The method of claim 2, wherein said compound is Compound 111.

68. The method of claim 2, wherein said compound is Compound 112.

69. The method of claim 2, wherein said compound is Compound 113.

70. The method of claim 2, wherein said compound is Compound 114.

71. The method of claim 2, wherein said compound is Compound 115.

72. The method of claim 2, wherein said compound is Compound 116.

73. The method of claim 2, wherein said compound is Compound 117.

74. The method of claim 2, wherein said compound is Compound 118.
